(19) United States
(12) Patent Application Publication
(10) Pub. No.: US 2002/0164663 A1
(43) Pub. Date: Nov. 7, 2002

(54) Methods and compositions for detection, diagnosis and prediction of antiestrogen-resistant breast cancer

(75) Inventors: Suzanne A. W. Fuqua, Sugarland, TX (US); William Friedrichs, Bergheim, TX (US)

Correspondence Address: Mark B. Wilson, FULBRIGHT & JAWORSKI L.L.P., Suite 2400, 600 Congress Avenue, Austin, TX, 78701, US

(73) Assignee: BOARD OF REGENTS, THE UNIVERSITY OF TEXAS SYSTEM, Austin, TX

(21) Appl. No.: 09/877,794

(22) Filed: Jun. 8, 2001

Related U.S. Application Data
(63) Continuation of application No. PCT/US99/28206, filed on Nov. 29, 1999.
(60) Provisional application No. 60/111,428, filed on Dec. 8, 1998.

(51) Int. Cl. G01N 33/574

(52) U.S. Cl. 435/7.23

(57) ABSTRACT

Disclosed are methods for the detection, diagnosis and prediction of tamoxifen-resistant breast cancer. Genetic and antibody probes and methods useful in determining the presence and monitoring the progression of breast cancer are also described. The methods involve determining polypeptide or mRNA expression of the genes encoding the angiogenic agents or receptors TIE-2, EDNRA, TGFβ3, TGFRβIII, VEGFR1, VEGF or bFGFR. Also described are procedures for combination therapies utilizing antiangiogenic agents or gene therapy directed towards TIE-2, EDNRA, TGFβ3, TGFRβIII, VEGFR1, VEGF or bFGFR, in combination with tamoxifen treatment of breast cancer.

The United States government has rights to use the present invention pursuant to Grant No. #PHS P50 CA58183-05, from the National Institutes of Health.

### BACKGROUND OF THE INVENTION

1. Field of the Invention

The present invention relates generally to methods of detecting antiestrogen resistant human breast cancer and the use of polypeptides and nucleic acids encoding angiogenic factors or angiogenic receptors for such methods. More particularly, certain methods utilizing differential expression of genes encoding tyrosine protein kinase receptor (TIE-2), endothelin-1 receptor (EDNRA), transforming growth factor β3 (TGFβ3), transforming growth factor receptor βIII (TGFRβIII), vascular permeability factor receptor (VEGFR1), vascular endothelin growth factor (VEGF) and basic fibroblast growth factor receptor (bFGFR) are described that may provide the basis for predictive and diagnostic evaluations of human breast cancer patients.

2. Description of Related Art

Breast cancer is the leading cause of death for women between 30-50 years of age in the United States. Pathological breast cancer staging (tumor size, nodal status) is still the most reliable method for predicting outcome. In contrast to other forms of cancer, only a few tumor markers have been identified for breast cancer (e.g., estrogen receptor, progesterone receptor, S-phase, P53, Erb-2, cathepsin D) (see, e.g. Slamon et al., 1987).

Mutational analysis of important tumor suppressor genes such as p53 (Elledge, 1994) and BRCA1 (Miki et al., 1994) has recently been introduced as a diagnostic and prognostic test for breast cancer. Mutations in the breast cancer susceptibility genes BRCA1 (chromosome 17q21) and BRCA2 (chromosome 13q13) are associated with familial breast cancer, accounting for about 5% of total breast cancer cases, but have not been found in sporadic breast cancer (Stratton and Wooster, 1996). To date, none of these markers has proven to be reliable enough to be used for routine screening for breast cancer in the clinic. Therefore, there is an urgent need for better prognostic markers in breast cancer diagnosis, measured either by “traditional” methods (e.g., immunohistochemistry, Western blot), or genetic test.

Tamoxifen is the most commonly prescribed drug for breast cancer in the world (Johnston, 1997). Tamoxifen is thought to inhibit breast cancer growth by competitively blocking the estrogen receptor (ER), thereby inhibiting estrogen-induced growth (Osborne and Fuqua, 1994). Over the past two decades its role has expanded from primary treatment for advanced metastatic disease to established adjuvant therapy following surgery for primary disease (Johnston, 1997). Tamoxifen prolongs both disease-free and overall survival in breast cancer patients (Johnston, 1997). But, while tamoxifen is effective in many breast cancer patients, eventually all patients develop tamoxifen resistance (Johnston, 1997). Thus, the widespread use of tamoxifen in clinical practice has resulted in a significant increase in the number of patients presenting at recurrence with tamoxifen-resistant disease (Johnston, 1997). The mechanisms for tamoxifen resistance are largely unknown and their identification could have profound clinical implications for alternative treatment strategies (Osborne and Fuqua, 1994; Johnston, 1997).

Previous studies in the areas of tamoxifen resistance and breast cancer progression have focused on alterations in the estrogen receptor (Osborne and Fuqua, 1994; lemieux and Fuqua, 1996; Zhang et al., 1997a), changes in ER accessory proteins (Osborne & Fuqua, 1994), clonal selection of ER negative tumor cells (Johnston, 1997), apoptosis factors (Johnston, 1997), AP-1 (Schiff et al., 1998), SRC-1 (Berns et al., 1998) and growth factor receptors (Johnston, 1997). It has been reported that overexpression of single growth factor genes such as cyclin D1 (Neuman et al., 1997), protein kinase A (Fujimoto and Katzenellenbogen, 1994) and transforming growth factor β (Thompson et al., 1991) can influence a cell's response to tamoxifen treatment. Despite this extensive work, the precise mechanisms underlying acquired tamoxifen resistance remain poorly understood.

Breast cancer is a heterogeneous disease and the development of tamoxifen resistance is probably multifactorial (Osborne and Fuqua, 1994). Thus, complex changes in patterns of gene expression may accompany the resistant phenotype. The present invention satisfies a long-standing need in the field by identifying changes in gene expression that are associated with the development of tamoxifen resistance.

Those genes identified herein as differentially expressed during the development of tamoxifen resistance generally fall into the categories of angiogenic factors or angiogenic receptors. An association between angiogenesis and tumor growth has been reported and anticancer therapies based upon antiangiogenic agents have been explored (Folkman, 1995a; Lin et al., 1998). However, the present application is the first report of an association between the development of tamoxifen resistance and the differential expression of angiogenic factors or receptors in human cancer.

### SUMMARY OF THE INVENTION

The present invention addresses deficiencies in the art by identifying specific gene products whose expression levels serve as markers for tamoxifen-resistant breast cancer. More particularly, differential expression of the genes encoding tyrosine protein kinase receptor (TIE-2, GenBank Accession No. L06139), endothelin-1 receptor (EDNRA, GenBank Accession No. L06622), transforming growth factor β3 (TGFβ3, GenBank Accession No. J03241), transforming growth factor receptor β3 (TGFRβIII, GenBank Accession No. L07594), vascular permeability factor receptor (VEGFR1, GenBank Accession No. U01134), vascular endothelin growth factor (VEGF, GenBank Accession Nos. M32977) and basic fibroblast growth factor receptor (bFGFR, GenBank Accession No. M60485) are reported herein to be associated with tamoxifen-resistant breast cancer. This surprising result is the first report of an association between the development of tamoxifen-resistant tumors and changes in expression of angiogenic factors or receptors. These results provide the basis for methods directed toward detection of expression levels of TIE-2, EDNRA, TGFβ3, TGFRβIII, VEGFR1, VEGF and bFGFR in breast tissue samples which will have utility for diagnosis and prediction of tamoxifen-resistant breast cancer.

One aspect of the present invention encompasses antibodies specific for TIE-2, EDNRA, TGFβ3, TGFRβIII, VEGFR1, VEGF and bFGFR and immunological methods for detection and measurement of these proteins in tissue samples. Such methods may include the use of Western blots, immunohistochemistry (IHC), ELISA, and other well known techniques for antibody assay of protein expression. Another aspect concerns the use of such antibodies for methods of breast cancer cell detection, diagnosis and prediction, by comparing the levels of for TIE-2, EDNRA, TGFβ3, TGFRβIII, VEGFR1, VEGF and bFGFR polypeptide in suspected tamoxifen-resistant cancer cells with levels present in groups of known estrogen stimulated, tamoxifen-sensitive and tamoxifen-resistant breast cancer cells.

One embodiment of the invention encompasses a kit for use in the detection and measurement of these proteins in tissue samples, comprising antibodies specific for TIE-2, EDNRA, TGFβ3, TGFRβIII, VEGFR1, VEGF or bFGFR. Additional components of kits for immunologic detection of disease-state associated antigens are well known in the art, and may include components such as molecular weight marker proteins, secondary antibodies, reagents for staining or otherwise detecting bound antibodies, control samples containing known amounts of TIE-2, EDNRA, TGFβ3, TGFRβIII, VEGFR1, VEGF and bFGFR protein or peptide, and negative controls lacking these proteins.

The invention also comprises nucleic acid segments that are either identical to or complementary with the cDNA sequences of TIE-2, EDNRA, TGFβ3, TGFRβIII, VEGFR1, VEGF and bFGFR. Such nucleic acid segments are expected to have utility not only as probes or primers for the genetic analysis of breast tumor samples but also, for example, as components of expression vectors or antisense vectors for transformation of tamoxifen-resistant breast cancer cells that differentially express these proteins. Such vectors may have utility in the treatment of tamoxifen-resistant breast cancer.

An additional embodiment encompasses genetic analysis of tissue samples to obtain information relating to tumor progression and tamoxifen-resistance. Such analyses typically employ PCRT amplification, using primers specific for the human cDNA sequences of TIE-2, EDNRA, TGFβ3, TGFRβIII, VEGFR1, VEGF and bFGFR, followed by quantitative analysis of the amplification products. Quantitative analysis of amplification products or of the mRNA species themselves may be performed by any standard means, including Southern blots, slot-blots, and Northern blots. In a preferred embodiment, the mRNA species present in a tissue sample are converted to cDNA prior to amplification, using reverse transcriptase. One example of such a protocol is the well known procedure of RT-PCR™. Tumors with differentially expressed levels of TIE-2, EDNRA, TGFβ3, TGFRβIII, VEGFR1, VEGF and bFGFR are recognized as associated with a poorer five-year survival rate for breast cancer patients. One may therefore assess potential survival rates in such patients by assaying the levels of these mRNA or protein species.

Yet another aspect of the present invention encompasses host cells or vectors comprising a nucleic acid encoding TIE-2, EDNRA, TGFβ3, TGFRβIII, VEGFR1, VEGF or bFGFR. Such cells or vectors are expected to have utility in the therapeutic treatment of breast cancer. Insertion of a vector comprising an antisense TIE-2, EDNRA, TGFβ3, TGFRβIII or VEGFR1, or an expression cassette for VEGF or bFGFR into tumor cells from breast cancers may result in suppression of tumor growth and colony formation. Thus, an embodiment of the present invention comprises a method for altering the phenotype of a tumor cell by contacting the cell with a nucleic acid encoding antisense or expression cassettes for TIE-2, EDNRA, TGFβ3, TGFRβIII, VEGFR1, VEGF or bFGFR, operably linked to a functional promoter, under conditions permitting uptake and expression of the nucleic acid by the tumor cell.

A further embodiment of the present invention concerns the use of antiangiogenic agents or gene therapy as an adjunct to tamoxifen treatment, or to convert tamoxifen resistant tumors into tamoxifen sensitive tumors. Antiantiogenic gene therapy may be accomplished, for example, by the methods of Lin et al., (1998), incorporated herein by reference in its entirety. Alternatively, antiangiogenic agents, such as AGM-1470 (TNP-470), platelet factor 4 and angiostatin may be used as tamoxifen adjuncts or for conversion of tamoxifen-resistant to tamoxifen-sensitive tumors (Folkman, 1995b). Additional antiangiogenic agents that may be used in the practice of the present invention are identified in Augustin (1998), incorporated herein by reference in its entirety. Antiangiogenic therapy may be combined with traditional forms of chemotherapy or radiation therapy (Folkman, 1995a), targeted specifically against tamoxifen-resistant breast tumors.

### BRIEF DESCRIPTION OF THE DRAWINGS

FIG. 1 shows a scatterplot matrix of expression data for 588 genes, collected from estrogen-stimulated (ES), tamoxifen-sensitive (TS) and tamoxifen-resistant (TR) breast cancers. Data were collected as described in the EXAMPLES section.

FIG. 2A shows a scatterplot of log-transformed expression data for TS and TR tumors, showing the line of identity (solid line) and 99% prediction region (dashed line). Genes that are overexpressed in TR tumors compared to TS tumors are indicated by open circles and underexpressed genes are indicated by solid triangles.

FIG. 2B shows a scatterplot of first and second principal components from the same data as shown in FIG. 2A.

FIG. 3 illustrates a scatterplot of second and third principal components from PCA (principal component analysis) of log-transformed gene expression data from ES, TS and TR tumors, back transformed to show approximate fold alterations. Axis labels describe the qualitative interpretation of PCA coefficients. Genes inside the 99% prediction ellipse (indicated by solid line) are shown as open circles, genes outside the ellipse are shown as closed circles.

FIG. 4 shows a Western blot analysis with erk-2 and HSF-1 antibodies in ES, TS and TR tumors. Molecular weight marker positions are indicated on the right side.

FIG. 5 illustrates the fold change in expression in estrogen-stimulated (E2), tamoxifen-sensitive (TS) and tamoxifen-resistant (TR) breast cancers for the TGFRβIII, VEGR1, TGFβ3, EDNRA and TIE-2 genes. Data were collected as described in the EXAMPLES section.

FIG. 6 describes the fold change in expression in estrogen-stimulated (E2), tamoxifen-sensitive (TS) and tamoxifen-resistant (TR) breast cancers for the VEGF and bFGFR genes, as described in the legend to FIG. 5.

FIG. 7 shows a Western blot analysis using a commercial antibody (Santa Cruz, Inc., Santa Cruz, Calif.) to the TIE-2 receptor protein. Five tumors of each group (E2, TS and TR) were examined. Only the TR tumors exhibited detectable expression of a high molecular weight (220 kDa) form of TIE-2 (putative TIE-2 related protein).

FIG. 8 shows a Western blot analysis using a commercial antibody (Santa Cruz, Inc., Santa Cruz, Calif.) to the TIE-2 receptor protein. Human vascular endothelial cells (HuVec) and one tumor of each mouse breast cancer group (E2, TS and TR) were examined. HuVec cells express a TIE-2 protein of approximately 140 kDa, compared to the 220 kDa TIE-2 related protein expressed in TR tumors.

FIG. 9 shows a Western blot analysis using an antibody to the VEGF protein. Five tumors of each group (E2, TS and TR) were examined. VEGF monomer and dimers were relatively overexpressed in the TR tumors.

### DETAILED DESCRIPTION OF THE PREFERRED EMBODIMENTS

This application concerns, at least in part, isolated proteins and nucleic acids encoded by tyrosine protein kinase receptor (TIE-2, GenBank Accession No. L06139), endothelin-1 receptor (EDNRA, GenBank Accession No. L06622), transforming growth factor β3 (TGFβ3, GenBank Accession No. J03241), transforming growth factor receptor βIII (TGFRβIII, GenBank Accession No. L07594), vascular permeability factor receptor (VEGFR1, GenBank Accession No. U01134), vascular endothelin growth factor (VEGF, GenBank Accession Nos. M32977) and basic fibroblast growth factor receptor (bFGFR, GenBank Accession No. M60485) as well as methods of detection, diagnosis, prediction and therapeutic treatment of tamoxifen-resistant breast cancer directed towards such proteins and nucleic acids.

Proteins

In referring to the function of TIE-2, EDNRA, TGFβ3, TGFRβIII, VEGFR1, VEGF or bFGFR or “wild-type” activity, it is meant that the molecule in question has the ability to inhibit angiogenesis, or to prevent metastasis or invasive tumor growth. Molecules possessing this activity may be identified using assays familiar to those of skill in the art. For example, transfer of genes encoding TIE-2, EDNRA, TGFβ3, TGFRβIII, VEGFR1, VEGF or bFGFR, or variants thereof, into cells that do not have a functional product, and hence exhibit impaired growth control, will identify, by virtue of growth suppression, those molecules having TIE-2, EDNRA, TGFβ3, TGFRβIII, VEGFR1, VEGF or bFGFR function.

The term “TIE-2, EDNRA, TGFβ3, TGFRβIII, VEGFR1, VEGF or bFGFR gene” refers to any DNA sequence that is substantially identical to a DNA sequence encoding a TIE-2, EDNRA, TGFβ3, TGFRβIII, VEGFR1, VEGF or bFGFR protein as defined above. Allowing for the degeneracy of the genetic code, sequences that have at least about 50%, usually at least about 60%, more usually about 70%, most usually about 80%, preferably at least about 90%, and most preferably about 95% of nucleotides that are identical to the cDNA sequences of TIE-2, EDNRA, TGFβ3, TGFRβIII, VEGFR1, VEGF or bFGFR are “as set forth in” those sequences. Sequences that are substantially identical or “essentially the same” as the cDNA sequences of TIE-2, EDNRA, TGFβ3, TGFRβIII, VEGFR1, VEGF or bFGFR also may be functionally defined as sequences that are capable of hybridizing to a nucleic acid segment containing the complement of the cDNA sequences of TIE-2, EDNRA, TGFβ3, TGFRβIII, VEGFR1, VEGF or bFGFR under conditions of relatively high stringency. Such conditions are typically relatively low salt and/or high temperature conditions, such as provided by about 0.02 M to about 0.15 M NaCl at temperatures of about 50° C. to about 70° C. Such selective conditions tolerate little, if any, mismatch between the complementary stands and the template or target strand. “TIE-2, EDNRA, TGFβ3, TGFRβIII, VEGFR1, VEGF or bFGFR gene” is also intended to include RNA, or antisense sequences compatible with the cDNA sequences. Any such gene sequences may also comprise associated control sequences.

The term “substantially identical,” when used to define either an amino acid sequence or a nucleic acid sequence, means that a particular subject sequence, for example, a mutant sequence, varies from the sequence of the natural TIE-2, EDNRA, TGFβ3, TGFRβIII, VEGFR1, VEGF or bFGFR genes by one or more substitutions, deletions, or additions, the net effect of which is to retain at least some biological activity of the protein or gene.

Alternatively, DNA analog sequences are “substantially identical” to specific DNA sequences disclosed herein if: (a) the DNA analog sequence is derived from coding regions of the natural TIE-2, EDNRA, TGFβ3, TGFRβIII, VEGFR1, VEGF or bFGFR gene; or (b) the DNA analog sequence is capable of hybridization of DNA sequences of (a) under moderately stringent conditions and which encode biologically active TIE-2, EDNRA, TGFβ3, TGFRβIII, VEGFR1, VEGF or bFGFR; or (c) DNA sequences which are degenerative as a result of the genetic code to the DNA analog sequences defined in (a) or (b).

The present invention also relates to fragments of the polypeptides that may or may not retain the angiogenic (or other) activity of TIE-2, EDNRA, TGFβ3, TGFRβIII, VEGFR1, VEGF or bFGFR. Fragments including the N-terminus of the molecule may be generated by genetic engineering of translation stop sites within the coding region (discussed below). Alternatively, treatment of the protein molecule with proteolytic enzymes, known as proteases, can produce a variety of N-terminal, C-terminal and internal fragments. Examples of fragments may include contiguous residues of the TIE-2, EDNRA, TGFβ3, TGFRβIII, VEGFR1, VEGF or bFGFR amino acid sequences of 6, 7, 8, 9, 10, 11, 12, 13, 14, 15, 16, 17, 18, 19, 20, 21, 22, 23, 24, 25, 30, 35, 40, 45, 50, 55, 60, 65, 75, 80, 85, 90, 95, 100, 200, 300, 400, or more amino acids in length. These fragments may be purified according to known methods, such as precipitation (e.g., ammonium sulfate), HPLC, ion exchange chromatography, affinity chromatography (including immunoaffinity chromatography), or various size separations (e.g., sedimentation, gel electrophoresis, gel filtration).

Substantially identical analog proteins will be greater than about 80% similar to the corresponding sequence of the native protein. Sequences having lesser degrees of similarity but comparable biological activity are considered to be equivalents. In determining nucleic acid sequences, all subject nucleic acid sequences capable of encoding substantially similar amino acid sequences are considered to be substantially similar to a reference nucleic acid sequence, regardless of differences in codon sequence.

Purification of Proteins

It may be desirable to purify TIE-2, EDNRA, TGFβ3, TGFRβIII, VEGFR1, VEGF or bFGFR or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, gel exclusion chromatography, polyacrylamide gel electrophoresis, affinity chromatography, immunoaffinity chromatography and isoelectric focusing. A particularly efficient method of purifying peptides is fast protein liquid chromatography (FPLC) or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide, therefore, also refers to a protein or peptide free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95%, or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “-fold purification number.” The actual units used to represent the amount of activity will, of course, be dependent upon the particular assay technique chosen to follow the purification, and whether or not the expressed protein or peptide exhibits a detectable activity.

Various techniques suitable for use in protein purification will be well known to those of skill in the art. These include, for example, precipitation with ammonium sulphate, PEG, antibodies and the like, or by heat denaturation, followed by: centrifugation; chromatography steps such as ion exchange, gel filtration, reverse phase, hydroxylapatite and affinity chromatography; isoelectric focusing; gel electrophoresis; and combinations of these and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will, therefore, be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of min, or at most an h. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size of the pores. Hence, molecules are eluted from the column in decreasing size, so long as the shape is relatively constant. Gel chromatography is unsurpassed for separating molecules of different size because separation is independent of all other factors such as pH, ionic strength, temperature, etc. Thus the elution volume is related in a simple matter to molecular weight.

Affinity chromatography is a chromatographic procedure that relies on the specific affinity between a substance to be isolated and a molecule to which it can specifically bind to. This is a receptor-ligand type of interaction. The column material is synthesized by covalently coupling one of the binding partners to an insoluble matrix. The column material is then able to specifically adsorb the substance from the solution. Elution occurs by changing the conditions to those in which binding will not occur (e.g., altered pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

Synthetic Peptides

The present invention also describes smaller TIE-2, EDNRA, TGFβ3, TGFRβIII, VEGFR1, VEGF or bFGFR peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to selected regions of the TIE-2, EDNRA, TGFβ3, TGFRβIII, VEGFR1, VEGF or bFGFR proteins, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell, and cultivated under conditions suitable for expression.

Antigen Compositions

The present invention also provides for the use of TIE-2, EDNRA, TGFβ3, TGFRβIII, VEGFR 1, VEGF or bFGFR proteins or peptides as antigens for the immunization of animals relating to the production of antibodies. It is envisioned that either TIE-2, EDNRA, TGFβ3, TGFRβIII, VEGFR1, VEGF or bFGFR proteins, or portions thereof, will be coupled, bonded, bound, conjugated, or chemically-linked to one or more agents via linkers, polylinkers, or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyanin (KLH) or bovine serum albumin (BSA).

Nucleic Acids

The present invention also provides, in another embodiment, genes encoding TIE-2, EDNRA, TGFβ3, TGFRβIII, VEGFR1, VEGF or bFGFR. As discussed below, a “TIE-2, EDNRA, TGFβ3, TGFRβIII, VEGFR1, VEGF or bFGFR gene” may contain a variety of different bases and yet still produce a corresponding polypeptide that is indistinguishable functionally, and in some cases structurally, from the genes disclosed herein.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate, or enhance the function of TIE-2, EDNRA, TGFβ3, TGFRβIII, VEGFR1, VEGF or bFGFR.

Nucleic Acids Encoding TIE-2, EDNRA, TGFβ3, TGFRβIII, VEGFR1, VEGF or bFGFR

Nucleic acids according to the present invention may encode an entire gene, a domain of TIE-2, EDNRA, TGFβ3, TGFRβIII, VEGFR1, VEGF or bFGFR that expresses a tumor suppressing function, or any other fragment of the sequences set forth herein. The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that TIE-2, EDNRA, TGFβ3, TGFRβIII, VEGFR1, VEGF or bFGFR may be represented by natural variants that have slightly different nucleic acid sequences but, nonetheless, encode the same proteins (see Table 1 below).

As used in this application, the term “a nucleic acid encoding a TIE-2, EDNRA, TGFβ3, TGFRβIII, VEGFR1, VEGF or bFGFR” refers to a nucleic acid molecule that has been isolated free of total cellular nucleic acid. The term “functionally equivalent codon” is used herein to refer to codons that encode the same amino acid, such as the six codons for arginine or serine (Table 1, below), and also refers to codons that encode biologically equivalent amino acids, as discussed in the following pages.

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG |
| | | | CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG |
| | | | CGU |
| Serine | Ser | S | AGC AGU UCA UCC UCG |
| | | | UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

The DNA segments of the present invention include those encoding biologically functional equivalent TIE-2, EDNRA, TGFβ3, TGFRβIII, VEGFR 1, VEGF or bFGFR proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

Oligonucleotide Probes and Primers

Naturally, the present invention also encompasses DNA segments that are complementary, or essentially complementary, to the cDNA sequences of TIE-2, EDNRA, TGFβ3, TGFRβIII, VEGFR1, VEGF or bFGFR. Nucleic acid sequences that are “complementary” are those that are capable of base-pairing according to the standard Watson-Crick complementary rules. As used herein, the term “complementary sequences” means nucleic acid sequences that are complementary to the extent that they are capable of hybridizing under relatively stringent conditions such as those described herein. Such sequences may encode the entire TIE-2, EDNRA, TGFβ3, TGFRβIII, VEGFR1, VEGF or bFGFR protein or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100, or more base pairs will be used, although others are contemplated. Longer polynucleotides encoding 250, 500, 1000, 1212, 1500, 2000, 2500, 3000, or 3040 bases and longer are contemplated as well. Such oligonucleotides will find use, for example, as probes in Southern and Northern blots and as primers in amplification reactions.

Hybridization Conditions

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl, at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS (sodium dodecylsulphate) also may be used to alter the hybridization conditions.

One method of using probes and primers of the present invention is in the search for genes related to TIE-2, EDNRA, TGFβ3, TGFRβIII, VEGFR1, VEGF or bFGFR. Normally, the target DNA will be a genomic or cDNA library, although screening may involve analysis of RNA molecules. By varying the stringency of hybridization, and the region of the probe, different degrees of homology may be discovered.

Antisense Constructs

Antisense technology may be used to “knock-out” function of TIE-2, EDNRA, TGFβ3, TGFRβIII, VEGFR1, VEGF or bFGFR in the treatment of tamoxifen-resistant breast cancers or in the development of cell lines or transgenic mice for research, diagnostic and screening purposes.

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription, or translation, or both within a host cell, either in vitro or in vivo, such as within a host animal, including a human subject.

Antisense constructs may be designed to bind to the promoter and other control regions, exons, introns, or even exon-intron boundaries of a gene. It is contemplated that the most effective antisense constructs will include regions complementary to intron/exon splice junctions. Thus, it is proposed that a preferred embodiment includes an antisense construct with complementarity to regions within about 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

Ribozymes

Another approach for addressing overexpression of genes in breast cancer is through the use of ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cell lines to which they were applied; the altered genes included the oncogenes H-ras, c-fos and genes of HIV. Most of this work involved the modification of a target mRNA, based on a specific mutant codon that is cleaved by a specific ribozyme.

It is anticipated that particularly appropriate targets for ribozyme or antisense directed therapies for tamoxifen-resistant breast cancer would be the genes or gene products for TIE-2, EDNRA, TGFβ3, TGFRβIII, VEGFR 1, VEGF or bFGFR.

Vectors for Cloning, Gene Transfer and Expression

Within certain embodiments expression vectors are employed to express the TIE-2, EDNRA, TGFβ3, TGFRβIII, VEGFR1, VEGF or bFGFR polypeptide product, which can then be purified and, for example, be used to vaccinate animals to generate antisera or monoclonal antibody with which further studies may be conducted. In other embodiments, the expression vectors are used in gene therapy.

Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Regulatory Elements

Throughout this application, the term “expression construct” is meant to include any type of genetic construct containing a nucleic acid coding for a gene product in which part or all of the nucleic acid coding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box. However, in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp before activity begins to decline. Depending on the promoter, it appears that individual elements can function either co-operatively or independently to activate transcription.

The particular promoter employed to control the expression of a nucleic acid sequence of interest is not believed to be important, so long as it is capable of directing the expression of the nucleic acid in the targeted cell. Thus, where a human cell is targeted, it is preferable to position the nucleic acid coding region adjacent and under the control of a promoter that is capable of being expressed in a human cell. Generally speaking, such a promoter might include either a human or viral promoter.

In various embodiments, the human cytomegalovirus (CMV) immediate early gene promoter, the SV40 early promoter, the Rous sarcoma virus long terminal repeat, rat insulin promoter, and glyceraldehyde-3-phosphate dehydrogenase promoter can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several elements/promoters which may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers, and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) also could be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2

ENHANCER/PROMOTER
Immunoglobulin Heavy Chain
Immunoglobulin Light Chain
T-Cell Receptor
HLA DQ α and DQ β
β-Interferon
Interleukin-2
Interleukin-2 Receptor
MHC Class II 5
MHC Class II HLA-DRα
β-Actin
Prealbumin (Transthyretin)
Muscle Creatine Kinase
Elastase I
Metallothionein
Collagenase
Albumin Gene
α-Fetoprotein
τ-Globin
β-Globin
e-fos
c-HA-ras
Insulin
Neural Cell Adhesion Molecule (NCAM)
α1-Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus

TABLE 3

| Element | Inducer |
|---|---|
| MT II | Phorbol Ester (TPA) |
| | Heavy metals |
| MMTV (mouse mammary tumor | Glucocorticoids |
| virus) | |
| β-Interferon | poly(rl)X, poly(rc) |
| Adenovirus 5 E2 | Ela |
| c-jun | Phorbol Ester (TPA), H₂O₂ |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease Virus |
| GRP78 Gene | A23187 |
| α-2-Macroglobulin | IL-6 |
| Vimentin | Serum |
| MHC Class I Gene H-2kB | Interferon |
| HSP70 | Ela, SV40 Large T Antigen |
| Proliferin | Phorbol Ester-TPA |
| Tumor Necrosis Factor | FMA |
| Thyroid Stimulating Hormone α | Thyroid Hormone |
| Gene | |
| Insulin E Box | Glucose |

Where a cDNA insert is employed, typically one will typically include a polyadenylation signal to effect proper polyadenylation of the gene transcript. The nature of the polyadenylation signal is not believed to be crucial to the successful practice of the invention, and any such sequence may be employed, such as human growth hormone and SV40 polyadenylation signals. Also contemplated as an element of the expression construct is a terminator. These elements can serve to enhance message levels and to minimize read through from the construct into other sequences.

Selectable Markers

In certain embodiments of the invention, the cells containing nucleic acid constructs of the present invention may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants. For example, genes that confer resistance to neomycin, puromycin, hygromycin, DHFR, GPT, zeocin, and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

Delivery of Expression Vectors

There are a number of ways in which expression vectors may introduced into cells. In certain embodiments of the invention, the expression construct comprises a virus or engineered construct derived from a viral genome. The ability of certain viruses to enter cells via receptor-mediated endocytosis, to integrate into a host cell genome, and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). Preferred gene therapy vectors are generally viral vectors.

Although some viruses that can accept foreign genetic material are limited in the number of nucleotides they can accommodate and in the range of cells they infect, these viruses have been demonstrated to successfully effect gene expression. However, adenoviruses do not integrate their genetic material into the host genome and therefore do not require host replication for gene expression making them ideally suited for rapid, efficient, heterologous gene expression. Techniques for preparing replication infective viruses are well known in the art.

Of course in using viral delivery systems, one will desire to purify the virion sufficiently to render it essentially free of undesirable contaminants, such as defective interfering viral particles or endotoxins and other pyrogens such that it will not cause any untoward reactions in the cell, animal or individual receiving the vector construct. A preferred means of purifying the vector involves the use of buoyant density gradients, such as cesium chloride gradient centrifugation.

Viruses used as gene vectors such as DNA viruses may include the papovaviruses (e.g., simian virus 40, bovine papilloma virus, and polyoma) (Ridgeway, 1988; Baichwal and Sugden, 1986) and adenoviruses (Ridgeway, 1988; Baichwal and Sugden, 1986).

One of the preferred methods for in vivo delivery involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to express an antisense polynucleotide that has been cloned therein.

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retroviral infection, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

In currently used systems, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of adenovirus vectors which are replication deficient depend on a unique helper cell line, designated 293, which is transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the E3, or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells, may be derived from the cells of other mammalian species that are permissive for human adenovirus. Such cells include, e.g., Vero cells or other monkey embryonic mesenchymal or epithelial cells. As discussed, the preferred helper cell line is 293.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) are employed as follows. A cell innoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking is initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking is commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

A typical vector applicable to practicing the present invention is replication defective and will not have an adenovirus E1 region. Thus, it will be most convenient to introduce the polynucleotide encoding the TIE-2, EDNRA, TGFβ3, TGFRβIII, VEGFR1, VEGF or bFGFR gene at the position from which the E1-coding sequences have been removed. However, the position of insertion of the construct within the adenovirus sequences is not critical. The polynucleotide encoding the TIE-2, EDNRA, TGFβ3, TGFRβIII, VEGFR1, VEGF or bFGFR gene may also be inserted in lieu of the deleted E3 region in E3 replacement vectors as described by Karlsson et al., (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

Other gene transfer vectors may be constructed from retroviruses. The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env. that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences, and also are required for integration in the host cell genome (Coffin, 1990).

In order to construct a retroviral vector, a nucleic acid encoding a TIE-2, EDNRA, TGFβ3, TGFRβIII, VEGFR1, VEGF or bFGFR gene is inserted into the viral genome in the place of certain viral sequences to produce a virus that is replication-defective. In order to produce virions, a packaging cell line containing the gag, pol, and env genes, but without the LTR and packaging components, is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are capable of infecting a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses has been designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, the infection of a variety of human cells that bear those surface antigens with an ecotropic virus in vitro was demonstrated (Roux et al., 1989).

There are certain limitations to the use of retrovirus vectors. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This may result from recombination events in which the intact sequence from the recombinant virus inserts upstream from the gag, pot, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs. Vectors derived from viruses such as vaccinia virus (Ridgeway, 1988; Baichwal and Sugden, 1986; Coupar et al., 1988), adeno-associated virus (AAV) (Ridgeway, 1988; Baichwal and Sugden, 1986; Hermonat and Muzycska, 1984), and herpes viruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight has been gained into the structure-function relationship of different viral sequences. In vitro studies showed that the virus could retain the ability for helper-dependent packaging and reverse transcription despite the deletion of up to 80% of its genome (Horwich et al., 1990). This suggests that large portions of the genome can be replaced with foreign genetic material. The hepatotropism and persistence (integration) are particularly attractive properties for liver-directed gene transfer. Chang et al. (1991) recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

To effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990), DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the TIE-2, EDNRA, TGFβ3, TGFRβIII, VEGFR1, VEGF or bFGFR gene may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a TIE-2, EDNRA, TGFβ3, TGFRβIII, VEGFR1, VEGF or bFGFR gene may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa, and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a TIE-2, EDNRA, TGFβ3, TGFRβIII, VEGFR1, VEGF or bFGFR gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a TIE-2, EDNRA, TGFβ3, TGFRβIII, VEGFR1, VEGF or bFGFR gene also may be specifically delivered into a cell type such as lung, epithelial, or tumor cells, by any number of receptor-ligand systems with or without liposomes. For example, epidermal growth factor (EGF) may be used as the receptor for mediated delivery of a nucleic acid encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia), and MAA (melanoma) can be used similarly as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

Primary mammalian cell cultures may be prepared in various ways. In order for the cells to be kept viable while in vitro and in contact with the expression construct, it is necessary to ensure that the cells maintain contact with the correct ratio of oxygen and carbon dioxide and nutrients but are protected from microbial contamination. Cell culture techniques are well documented and are disclosed herein by reference (Freshner, 1992).

Examples of useful mammalian host cell lines are Vero and HeLa cells and cell lines of Chinese hamster ovary, W138, BHK, COS-7, 293, HepG2, NIH3T3, RIN, and MDCK cells. In addition, a host cell strain may be chosen that modulates the expression of the inserted sequences, or modifies and processes the gene product in the manner desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to insure the correct modification and processing of the foreign protein expressed.

A number of selection systems may be used including, but not limited to, HSV thymidine kinase, hypoxanthine-guanine phosphoribosyltransferase and adenine phosphoribosyltransferase genes, in tk-, hgprt- or aprt- cells, respectively. Also, anti-metabolite resistance can be used as the basis of selection for dhfr: that confers resistance to methotrexate; gpt, that confers resistance to mycophenolic acid; neo, that confers resistance to the aminoglycoside G418; and hygro, that confers resistance to hygromycin.

Animal cells can be propagated in vitro in two modes: as non-anchorage dependent cells growing in suspension throughout the bulk of the culture or as anchorage-dependent cells requiring attachment to a solid substrate for their propagation (i.e., a monolayer type of cell growth).

Non-anchorage dependent or suspension cultures from continuous established cell lines are the most widely used means of large scale production of cells and cell products. However, suspension cultured cells have limitations, such as tumorigenic potential and lower protein production than adherent T-cells.

Large scale suspension culture of mammalian cells in stirred tanks is a common method for production of recombinant proteins. Two suspension culture reactor designs are in wide use—the stirred reactor and the airlift reactor. The stirred design has been used successfully on an 8000 liter capacity for the production of interferon. Cells are grown in a stainless steel tank with a height-to-diameter ratio of 1:1 to 3:1. The culture usually is mixed with one or more agitators, based on bladed disks or marine propeller patterns. Agitator systems offering less shear forces than blades have been described. Agitation may be driven either directly or indirectly by magnetically coupled drives. Indirect drives reduce the risk of microbial contamination through seals on stirrer shafts.

The airlift reactor, also initially described for microbial fermentation and later adapted for mammalian culture, relies on a gas stream to both mix and oxygenate the culture. The gas stream enters a riser section of the reactor and drives circulation. Gas disengages at the culture surface, causing denser liquid which is free of gas bubbles to travel downward in the downcomer section of the reactor. The main advantage of this design is the simplicity and lack of need for mechanical mixing. Typically, the height-to-diameter ratio is 10:1. The airlift reactor scales up relatively easily, has good mass transfer of gases and generates relatively low shear forces.

Generating Antibodies Reactive With TIE-2, EDNRA, TGFβ3, TGFRβIII, VEGFR1, VEGF or bFGFR

In another aspect, the present invention contemplates an antibody that is immunoreactive with a TIE-2, EDNRA, TGFβ3, TGFRβIII, VEGFR1, VEGF or bFGFR molecule of the present invention, or any portion thereof. An antibody can be a polyclonal or a monoclonal antibody. In a preferred embodiment, an antibody is a monoclonal antibody. Means for preparing and characterizing antibodies are well known in the art (see, e.g., Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal, for example, rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the antibodies of the present invention will find useful application in standard immunochemical procedures, such as ELISA and Western blot methods and in immunohistochemical procedures such as tissue staining, as well as in other procedures which may utilize antibodies specific to TIE-2, EDNRA, TGFβ3, TGFRβIII, VEGFR1, VEGF or bFGFR-related antigen epitopes.

The antibodies of the present invention are also useful for the isolation of TIE-2, EDNRA, TGFβ3, TGFRβIII, VEGFR1, VEGF or bFGFR polypeptides by immunoprecipitation. Immunoprecipitation involves the separation of the target antigen component from a complex mixture, and is used to discriminate or isolate minute amounts of protein. For the isolation of membrane proteins cells must be solubilized into detergent nicelles. Nonionic salts are preferred, since other agents such as bile salts, precipitate at acid pH or in the presence of bivalent cations. Antibodies are and their uses are discussed further, below.

In general, both polyclonal and monoclonal antibodies against TIE-2, EDNRA, TGFβ3, TGFRβIII, VEGFR1, VEGF or bFGFR may be used in a variety of embodiments. For example, they may be employed in antibody cloning protocols to obtain cDNAs or genes encoding other isoforms of TIE-2, EDNRA, TGFβ3, TGFRβIII, VEGFR1, VEGF or bFGFR or related proteins. They also may be used in inhibition studies to analyze the effects of TIE-2, EDNRA, TGFβ3, TGFRβIII, VEGFR1, VEGF or bFGFR-related peptides in cells or animals. A particularly useful application of such antibodies is in purifying native or recombinant TIE-2, EDNRA, TGFβ3, TGFRβIII, VEGFR1, VEGF or bFGFR, for example, using an antibody affinity column. The operation of all such immunological techniques will be known to those of skill in the art in light of the present disclosure.

Means for preparing and characterizing antibodies are well known in the art (see, e.g., Harlow and Lane, 1988; incorporated herein by reference). More specific examples of monoclonal antibody preparation are give in the examples below.

As is well known in the art, a given composition may vary in its immunogenicity. It is often necessary, therefore, to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin also can be used as carriers. Means for conjugating a polypeptide to a carrier protein are well known in the art and include glutaraldehyde, m-maleimidobenzoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection also may be given. The process of boosting and titering is repeated until a suitable titer is achieved. When a desired level of immunogenicity is obtained, the immunized animal can be bled and the serum isolated and stored, and/or the animal can be used to generate monoclonal antibodies.

Monoclonal antibodies may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified TIE-2, EDNRA, TGFβ3, TGFRβIII, VEGFR1, VEGF or bFGFR protein, polypeptide, or peptide or a cell expressing high levels of TIE-2, EDNRA, TGFβ3, TGFRβIII, VEGFR1, VEGF or bFGFR. The immunizing composition is administered in a manner effective to stimulate antibody producing cells. Cells from rodents such as mice and rats are preferred, however, the use of rabbit, sheep or frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XXO Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus (Kohler and Milstein, 1975; 1976), and those using polyethylene glycol (PEG), such as 37% (v/v) PEG, have been described by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two wk. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three wk) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mnAbs in high concentration. The individual cell lines also could be cultured in vitro, where the mAbs are naturally secreted into the culture medium from which they can be readily obtained in high concentrations. mAbs produced by either means may be further purified, if desired, using filtration, centrifugation, and various chromatographic methods such as HPLC or affinity chromatography.

Diagnosing Cancers Involving TIE-2, EDNRA, TGFβ3, TGFRβIII, VEGFR1, VEGF or bFGFR

The present inventors have determined that alterations in expression of TIE-2, EDNRA, TGFβ3, TGFRβIII, VEGFR1, VEGF or bFGFR are associated with tamoxifen-resistant breast cancer and may be associated with other malignancies. Therefore, TIE-2, EDNRA, TGFβ3, TGFRβIII, VEGFR1, VEGF or bFGFR mRNAs and the corresponding genes may be employed as a diagnostic or predictive indicator of cancer, particularly tamoxifen-resistant breast cancer.

Genetic Diagnosis

One embodiment of the instant invention comprises a method for detecting variation in the expression of TIE-2, EDNRA, TGFβ3, TGFRβIII, VEGFR1, VEGF or bFGFR. This may comprise determining the level of expression of TIE-2, EDNRA, TGFβ3, TGFRβIII, VEGFR1, VEGF or bFGFR or determining specific alterations in the expressed product in a biological sample. In particular, the present invention relates to the diagnosis or prediction of tamoxifen-resistant breast cancer.

The nucleic acid used in the disclosed methods is isolated from cells contained in a biological sample, according to standard methodologies (Sambrook et al., 1989). The nucleic acid may be genomic DNA or fractionated or whole cell RNA. Where RNA is used, it may be desirable to convert the RNA to a complementary DNA. In one embodiment, the RNA is whole cell RNA; in another embodiment, it is poly-A RNA. Normally, the nucleic acid is amplified.

Depending on the format, the specific nucleic acid of interest is identified directly in the sample using amplification or by hybridization with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label, or even via a system using electrical or thermal impulse signals (Affymax Technology; Bellus, 1994).

Following detection, one compares the results obtained from a patient with a sufficiently large reference group of normal patients, patients with tamoxifen-sensitive breast cancer and patients with tamoxifen-resistant breast cancer. In this way, it is possible to correlate the amount of TIE-2, EDNRA, TGFβ3, TGFRβIII, VEGFR1, VEGF or bFGFR detected with various clinical states, such as tamoxifen-resistance. In particular applications, such as breast cancers, it is contemplated that different levels of progression of breast cancer may be identified.

Various types of defects are to be identified. Thus, “alterations” should be read as including deletions, insertions, point mutations and duplications. Point mutations result in stop codons, frameshift mutations or amino acid substitutions. Somatic mutations are those occurring in non-germline tissues. Germ-line mutations can occur in reproductive tissue and are inherited. Mutations in and outside the coding region also may affect the amount of TIE-2, EDNRA, TGFβ3, TGFRβIII, VEGFR 1, VEGF or bFGFR produced, both by altering the transcription of the gene or in destabilizing or otherwise altering the processing of either the transcript (mRNA) or protein.

A variety of different assays are contemplated in this regard, including but not limited to, fluorescent in situ hybridization (FISH), direct DNA sequencing, PFGE analysis, Southern or Northern blotting, single-stranded conformation polymorphism (SSCP), RNAse protection assay, allele-specific oligonucleotide (ASO), dot blot analysis, denaturing gradient gel electrophoresis, RFLP and PCR™-SSCP.

An alternative method for detection of mutations in the TIE-2, EDNRA, TGFβ3, TGFRβIII, VEGFR1, VEGF or bFGFR sequences involves the recently developed protein truncation assay (PTT) to detect mutations affecting the length of the protein. This method is based on RT-PCR™ using an upstream PCR™ primer containing a RNA polymerase promoter and a eukaryotic translation initiation signal. Approximately 200 ng of the PCR™ product is used directly for the coupled in vitro transcription/translation reaction (coupled TNT T7 reticulocyte system, Promega) which is substituted with ³⁵S methionine. The amplified oligonucleotide products may be sequenced by standard techniques known to those skilled in the art.

Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to bind to the target DNA or RNA and need not be used in an amplification process.

In preferred embodiments, the probes or primers are labeled with radioactive species (³²P, ¹⁴C, ³⁵S, ³H, or other label), with a fluorophore (rhodamine, fluorescein), or a chemilluminescent moiety (luciferase).

Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site also may be useful in the amplification of nucleic acids in the present invention, Walker et al., (1992).

Strand Displacement Amplification (SDA) is another method of carrying out isothermal amplification of nucleic acids which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences also can be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still other amplification methods described in GB Application No. 2 202 328, and in PCT Application No. PCT/US89/01025, each of which is incorporated herein by reference in its entirety, may be used in accordance with the present invention. In the former application, “modified” primers are used in a PCR™-like, template- and enzyme-dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer, and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule having a sequence identical to that of the original RNA between the primers, and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR™” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide”, thereby amplifying the di-oligonucleotide, may also be used in the amplification step of the present invention as described in Wu et al., (1989), incorporated herein by reference in its entirety.

Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. (See Sambrook et al., 1989)

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

Detection Methods

Products may be visualized in order to confirm amplification of the marker sequences and to measure the relative amounts of amplification products as a measure of gene expression levels. One typical visualization method involves staining of a gel with ethidium bromide and visualization under UV light. Alternatively, if the amplification products are integrally labeled with radio- or fluorometrically-labeled nucleotides, the amplification products can then be exposed to X-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. (See Sambrook et al., 1989) For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. General techniques for determination of the DNA sequence of amplification products are well known in the art and include standard dideoxy sequencing by the Sanger technique (See Sambrook et al., 1989). Within certain methods, exhaustive analysis of genes is carried out by sequence analysis using primer sets designed for optimal sequencing (Pignon et al., 1994).

The present invention may utilize any or all of these types of analyses. Using the sequences disclosed herein, oligonucleotide primers, may be designed to permit the amplification of sequences throughout the TIE-2, EDNRA, TGFβ3, TGFRβIII, VEGFR1, VEGF or bFGFR genes that may then be analyzed by direct sequencing. The amplified sequences may also be identified and quantitated, using techniques well known in the art and further described herein. The expression levels of the TIE-2, EDNRA, TGFβ3, TGFRβIII, VEGFR1, VEGF or bFGFR genes or mutants thereof may be used in the methods disclosed herein to determine degree of malignancy, cell tumorigenicity, and potential diagnosis and prediction of cancers such as tamoxifen-resistant breast cancers.

Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by transfer of the separated nucleic acids (“blotting”) on to the filter.

Subsequently, the blotted target is incubated with a probe (usually labeled) under conditions that promote denaturation and rehybridization. Because the probe is designed to base pair with the target, the probe will bind a portion of the target sequence under renaturing conditions. Unbound probe is then removed, and the labeled probe detected and quantified using standard techniques known to those skilled in the art.

Kit Components

All the essential materials and reagents required for detecting, measuring, or sequencing TIE-2, EDNRA, TGFβ3, TGFRβIII, VEGFR1, VEGF or bFGFR and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

Chip Technologies

Specifically contemplated by the present inventors are chip-based DNA technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Chen et al., 1998); Pease et al. (1994); Fodor et al. (1991).

A preferred embodiment utilizes cDNA array technology, exemplified by the CLONTECH Atlas™ human cDNA expression array (CLONTECH Laboratories, Inc.). cDNA arrays offer the potential to simultaneously quantify expression of many genes. Advances in cDNA array technology to address array size, probe density, probe content and readout make this technology suitable for application in the laboratory (Marshall and Hodgson, 1998). However, the novelty of this technology means that there are no well-established and widely accepted standards to guide analysis and interpretation of the data. cDNA arrays have most often been utilized in paired comparisons (e.g. control vs. tumor) to identify differentially expressed genes in only a few types of cancer, such as melanoma (DeRisi et al., 1996), Ewing's sarcoma (Welford et al., 1998), alveolar rhabdomyosarcoma (Khan et al., 1998) and gastrointestinal tumors (Zhang et al., 1997). After standardization, rules for gene selection have typically been based on ratios of expression, for example, greater than two-fold difference (Schena et al., 1996), greater than three standard deviations of control genes ratio (DeRisi et al., 1996), or an arbitrary percent.

Due to expense, limited amounts of RNA and other considerations, array experiments have previously involved few replications and have orders of magnitude more variables (genes and ESTs) than observations. The study illustrated in the EXAMPLES section of the present disclosure shows the application of principal components analysis, coupled with robust estimates of 99% prediction regions or higher order components, as a practical approach to screening array data. The method presumes that the vast majority of genes will be altered very little and uses information from all genes to obtain more stable estimates of variability. The method is not limited to pairwise comparisons, but can be used to study several tumor types or experimental conditions simultaneously. This approach is capable of reliably identifying 60-85% of genes exhibiting moderate degrees of differential expression (2-2.5 fold) without increasing the number of spuriously identified outliers.

Immunodiagnosis

Antibodies of the present invention can be used in characterizing the TIE-2, EDNRA, TGFβ3, TGFRβIII, VEGFR1, VEGF or bFGFR content of healthy and diseased tissues, through techniques such as ELISA and Western blotting. This may provide a screen for the presence or absence of malignancy or as a predictor of cancer progression and patient survival.

The use of antibodies of the present invention, in an ELISA assay is contemplated. For example, anti-TIE-2, EDNRA, TGFβ3, TGFRβIII, VEGFR1, VEGF or bFGFR antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera, such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting the same to a second antibody having specificity for TIE-2, EDNRA, TGFβ3, TGFRβIII, VEGFR1, VEGF or bFGFR that differs from that of the first antibody. Appropriate conditions preferably include diluting the sample with diluents such as BSA, bovine gamma globulin (BGG), and phosphate buffered saline (PBS)/Tween®. These added agents also tend to assist in the reduction of nonspecific background. The layered antisera is then allowed to incubate for from about 2 to about 4 h, at temperatures preferably on the order of about 25° to about 27° C. Following incubation, the antisera-contacted surface is washed so as to remove non-immunocomplexed material. A preferred washing procedure includes washing with a solution such as PBS/Tween® or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-IgG for a period of time and under conditions which favor the development of immunocomplex formation (e.g., incubation for 2 h at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against TIE-2, EDNRA, TGFβ3, TGFRβIII, VEGFR1, VEGF or bFGFR proteins or the primary antibodies.

Methods for Screening Active Compounds

The present invention also contemplates the use of TIE-2, EDNRA, TGFβ3, TGFRβIII, VEGFR1, VEGF or bFGFR and active fragments, and nucleic acids coding therefor, in the screening of compounds for activity in blocking the effect of overexpression of these genes. These assays may make use of a variety of different formats and may depend on the kind of “activity” for which the screen is being conducted. Contemplated functional “read-outs” include binding to a compound, inhibition of binding to a substrate, ligand, receptor or other binding partner by a compound, phosphatase activity, anti-phosphatase activity, phosphorylation or dephosphorylation of TIE-2, EDNRA, TGFβ3, TGFRβIII, VEGFR1, VEGF or bFGFR, or inhibition or stimulation of angiogenesis, growth, metastasis, cell division, apoptosis, tumor progression or other malignant phenotype. Preferred embodiments include assay of cell replication by incorporation of radiolabeled thymidine or colony formation.

In Vitro Assays

In one embodiment, the invention is to be applied for the screening of compounds that bind to the TIE-2, EDNRA, TGFβ3, TGFRβIII, VEGFR1, VEGF or bFGFR molecules or a fragment thereof. The polypeptide or fragment may be either free in solution, fixed to a support, or expressed in or on the surface of a cell. Either the polypeptide or the compound may be labeled, thereby permitting the determination of binding.

In another embodiment, the assay may measure the inhibition of binding of TIE-2, EDNRA, TGFβ3, TGFRβIII, VEGFR1, VEGF or bFGFR to a natural or artificial substrate or binding partner. Competitive binding assays can be performed in which one of the agents is labeled. Usually, the polypeptide will be the labeled species. One may measure the amount of free label versus bound label to determine binding or inhibition of binding.

Another technique for high throughput screening of compounds is described in WO 84/03564, the contents of which are incorporated herein by reference. Large numbers of small peptide test compounds are synthesized on a solid substrate, such as plastic pins or some other surface. The peptide test compounds are reacted with TIE-2, EDNRA, TGFβ3, TGFRβIII, VEGFR1, VEGF or bFGFR and washed. Bound polypeptide is detected by various methods.

Purified TIE-2, EDNRA, TGFβ3, TGFRβIII, VEGFR1, VEGF or bFGFR can be coated directly onto plates for use in the aforementioned drug screening techniques. However, non-neutralizing antibodies to the polypeptide can be used to immobilize the polypeptide to a solid phase. Also, fusion proteins containing a reactive region (preferably a terminal region) may be used to link the TIE-2, EDNRA, TGFβ3, TGFRβIII, VEGFR1, VEGF or bFGFR active region to a solid phase.

Various cell lines containing wild-type or natural or engineered mutations in TIE-2, EDNRA, TGFβ3, TGFRβIII, VEGFR1, VEGF or bFGFR can be used to study various functional attributes of these proteins and how a candidate compound affects these attributes. Methods for engineering mutations are described elsewhere in this document. In such assays, the compound would be formulated appropriately, given its biochemical nature, and contacted with a target cell. Depending on the assay, culture may be required. The cell may then be examined by virtue of a number of different physiologic assays. Alternatively, molecular analysis may be performed in which the function of TIE-2, EDNRA, TGFβ3, TGFRβIII, VEGFR1, VEGF or bFGFR, or related pathways, may be explored. This may involve assays such as those for protein expression, enzyme function, substrate utilization, phosphorylation states of various molecules, cAMP levels, mRNA expression (including differential display of whole cell or polyA RNA) and others.

In Vivo Assays

The present invention also encompasses the use of various animal models. By developing or isolating mutant cells lines that show differential expression of TIE-2, EDNRA, TGFβ3, TGFRβIII, VEGFR1, VEGF or bFGFR, one can generate cancer models in mice that will be predictive of cancers in humans and other mammals. These models may employ the orthotopic or systemic administration of tumor cells to mimic primary and/or metastatic cancers. Alternatively, one may induce cancers in animals by providing agents known to be responsible for certain events associated with malignant transformation and/or tumor progression. Finally, transgenic animals (discussed below) that differentially express a wild-type TIE-2, EDNRA, TGFβ3, TGFRβIII, VEGFR1, VEGF or bFGFR may be utilized as models for cancer development and treatment.

Treatment of animals with test compounds will involve the administration of the compound, in an appropriate form, to the animal. Administration will be by any route that could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, survival, reduction of tumor burden or mass, arrest or slowing of tumor progression, elimination of tumors, inhibition or prevention of metastasis, increased activity level, improvement in immune effector function and improved food intake.

Rational Drug Design

The goal of rational drug design is to produce structural analogs of biologically active polypeptides or compounds with which they interact (agonists, antagonists, inhibitors, binding partners, etc.). By creating such analogs, it is possible to fashion drugs which are more active or stable than the natural molecules, which have different susceptibility to alteration or which may affect the function of various other molecules. In one approach, one would generate a three-dimensional structure for TIE-2, EDNRA, TGFβ3, TGFRβIII, VEGFR1, VEGF or bFGFR or a fragment thereof. This could be accomplished by x-ray crystallography, computer modeling or by a combination of both approaches. In addition, knowledge of the polypeptide sequences permits computer employed predictions of structure-function relationships. An alternative approach, an “alanine scan,” involves the random replacement of residues throughout a protein or peptide molecule with alanine, followed by determining the resulting effect(s) on protein function.

It also is possible to isolate a TIE-2, EDNRA, TGFβ3, TGFRβIII, VEGFR1, VEGF or bFGFR specific antibody, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of an anti-idiotype antibody would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs which have improved TIE-2, EDNRA, TGFβ3, TGFRβIII, VEGFR1, VEGF or bFGFR activity or which act as stimulators, inhibitors, agonists, or antagonists of TIE-2, EDNRA, TGFβ3, TGFRβIII, VEGFR1, VEGF or bFGFR or molecules affected by TIE-2, EDNRA, TGFβ3, TGFRβIII, VEGFR1, VEGF or bFGFR function.

Methods for Treating TIE-2, EDNRA, TGFβ3, TGFRβIII, VEGFR1, VEGF or bFGFR Related Malignancies

The present invention also contemplates, in another embodiment, the treatment of cancer. The types of cancer that may be treated, according to the present invention, are limited only by the involvement of TIE-2, EDNRA, TGFβ3, TGFRβIII, VEGFR1, VEGF or bFGFR. By involvement is meant that, it is not even a requirement that TIE-2, EDNRA, TGFβ3, TGFRβIII, VEGFR1, VEGF or bFGFR be mutated or abnormal—the overexpression or underexpression of these proteins may be a primary factor in the development of tamoxifen-resistance. Thus, it is contemplated that tumors may be treated using antisense or expression therapy targeted to TIE-2, EDNRA, TGFβ3, TGFRβIII, VEGFR1, VEGF or bFGFR.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

Genetic Based Therapies

One of the therapeutic embodiments contemplated by the present inventors is the intervention, at the molecular level, in the events involved in the tumorigenesis of some cancers. Specifically, the present inventors intend to provide, to a cancer cell, an antisense construct capable of inhibiting expression of TIE-2, EDNRA, TGFβ3, TGFRβIII, VEGFR1, VEGF or bFGFR, or an expression construct capable of increasing expression of VEGF or bFGFR in that cell. The lengthy discussion of expression vectors and the genetic elements employed therein is incorporated into this section by reference. Particularly preferred expression vectors are viral vectors such as adenovirus, adeno-associated virus, herpes virus, vaccinia virus and retrovirus. Also preferred is liposomally-encapsulated expression vector.

Those of skill in the art are well aware of how to apply gene delivery to in vivo and ex vivo situations. For viral vectors, one generally will prepare a viral vector stock. Depending on the kind of virus and the titer attainable, one will deliver between about 1×10⁴ and 1×10¹² infectious particles to the patient. Similar figures may be extrapolated for liposomal or other non-viral formulations by comparing relative uptake efficiencies. Formulation as a pharmaceutically acceptable composition is discussed below.

Various routes are contemplated for various tumor types. The section below on routes contains an extensive list of possible routes. For practically any tumor, systemic delivery is contemplated. This will prove especially important for attacking microscopic or metastatic cancer. Where discrete tumor mass may be identified, a variety of direct, local and regional approaches may be taken. For example, the tumor may be injected directly with the expression vector. A tumor bed may be treated prior to, during or after resection. Following resection, one generally will deliver the vector by a catheter left in place following surgery. One may utilize the tumor vasculature to introduce the vector into the tumor by injecting a supporting vein or artery. A more distal blood supply route also may be utilized.

In a different embodiment, ex vivo gene therapy is contemplated. This approach is particularly suited, although not limited, to treatment of bone marrow associated cancers. In an ex vivo embodiment, cells from the patient are removed and maintained outside the body for at least some period of time. During this period, a therapy is delivered, after which the cells are reintroduced into the patient. Preferably, any tumor cells in the sample have been killed.

Immunotherapies

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

According to the present invention, native or wild type TIE-2, EDNRA, TGFβ3, TGFRβIII, VEGFR1, VEGF or bFGFR may be likely targets for an immune effector. It is possible TIE-2, EDNRA, TGFβ3, TGFRβIII, VEGFR1, VEGF or bFGFR may be targeted by immunotherapy, either using antibodies, antibody conjugates, or immune effector cells.

Alternatively, immunotherapy could be used as part of a combined therapy, in conjunction with TIE-2, EDNRA, TGFβ3, TGFRβIII, VEGFR1, VEGF or bFGFR-targeted gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, sialyl Lewis antigen, MucA, MucB, PLAP, estrogen receptor, laminin receptor, erb B and p155.

Combined Therapy With Immunotherapy, Traditional Chemo- or Radiotherapy

Tumor cell resistance to DNA damaging agents represents a major problem in clinical oncology. One goal of current cancer research is to find ways to improve the efficacy of chemo- and radiotherapy. One way is by combining such traditional therapies with gene therapy. For example, the herpes simplex-thymidine kinase (HS-tk) gene, when delivered to brain tumors by a retroviral vector system, successfully induced susceptibility to the antiviral agent ganciclovir (Culver et al., 1992). In the context of the present invention, it is contemplated that TIE-2, EDNRA, TGFβ3, TGFRβIII, VEGFR1, VEGF or bFGFR gene therapy could be used similarly in conjunction with chemo- or radiotherapeutic intervention.

To kill cells, inhibit cell growth, inhibit metastasis, inhibit angiogenesis or otherwise reverse or reduce the malignant phenotype of tumor cells, using the methods and compositions of the present invention, one would generally contact a “target” cell with an antisense construct of TIE-2, EDNRA, TGFβ3, TGFRβIII, VEGFR1, VEGF or bFGFR, or an expression construct of VEGF or bFGFR and at least one other agent. These compositions would be provided in a combined amount effective to kill or inhibit proliferation of the cell. This process may involve contacting the cells with the antisense or expression construct and the agent(s) or factor(s) at the same time. This may be achieved by contacting the cell with a single composition or pharmacological formulation that includes both agents, or by contacting the cell with two distinct compositions or formulations simultaneously, wherein one composition includes the antisense or expression construct and the other includes the agent.

Alternatively, the gene therapy treatment may precede or follow the other agent treatment by intervals ranging from min to wk. In embodiments where the other agent and expression construct are applied separately to the cell, one would generally ensure that a significant period of time did not expire between the time of each delivery, such that the agent and expression construct would still be able to exert an advantageously combined (e.g., synergistic) effect on the cell. In such instances, it is contemplated that one would contact the cell with both modalities within about 12-24 h of each other and, more preferably, within about 6-12 h of each other, with a delay time of only about 12 h being most preferred. In some situations, it may be desirable to extend the duration of treatment with only the therapeutic agent significantly, for example, where several days (2, 3, 4, 5, 6 or 7) to several wk (1, 2, 3, 4, 5, 6, 7 or 8) lapse between the respective administrations.

It also is conceivable that more than one administration of either TIE-2, EDNRA, TGFβ3, TGFRβIII, VEGFR1, VEGF or bFGFR or the other agent will be desired. Various combinations may be employed, where TIE-2, EDNRA, TGFβ3, TGFRβIII, VEGFR1, VEGF or bFGFR is “A” and the other agent is “B”, as exemplified below:

```
  A/B/A B/A/B B/B/A A/A/B B/A/A A/B/B B/B/B/A B/B/A/B
  A/A/B/B A/B/A/B A/B/B/A B/B/A/A B/A/B/A B/A/A/B B/B/B/A
  A/A/A/B B/A/A/A A/B/A/A A/A/B/A A/B/B/B B/A/B/B B/B/A/B
```

In addition, other combinations are contemplated. For instance, constructs targeted to two or more of the TIE-2, EDNRA, TGFβ3, TGFRβIII, VEGFR1, VEGF or bFGFR genes may be employed simultaneously to achieve an improved antiangiogenic effect. In a preferred embodiment, the agent “B” would comprise tamoxifen. To achieve cell killing, both agents are delivered to a cell in a combined amount effective to kill the cell.

Agents or factors suitable for use in a combined therapy are any chemical compound or treatment method that induces DNA damage when applied to a cell. Such agents and factors include radiation and waves that induce DNA damage such as β-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use include, e.g., adriamycin, 5-fluorouracil (5FU), etoposide (VP-16), camptothecin, actinomycin-D, mitomycin C cisplatin (CDDP) and even hydrogen peroxide. The invention also encompasses the use of a combination of one or more DNA damaging agents, whether radiation-based or actual compounds, such as the use of X-rays with cisplatin or the use of cisplatin with etoposide.

Particularly prefered for this embodiment is adjunct therapy with compounds that have reported antiangiogenic activity, such as angiotensin, laminin peptides, fibronectin peptides, plasminogen activator inhibitors, tissue metalloproteinase inhibitors, interferons, interleukin 12, platelet factor 4, IP-10, Gro-β, thrombospondin, 2-methoxyoestradiol, proliferin-related protein, carboxiamidotriazole, CM101, Marimastat, pentosan polysulphate, angiopoietin 2 (Regeneron), interferon-alpha, herbimycin A, PNU145156E, 16K prolactin fragment, Linomide, thalidomide, pentoxifylline, genistein, TNP-470, endostatin, paclitaxel, accutin, angiostatin, cidofovir, vincristine, bleomycin, AGM-1470, platelet factor 4 or minocycline. It is anticipated that such agents may be used in combination with either tamoxifen therapy and/or gene therapy targeted to TIE-2, EDNRA, TGFβ3, TGFRβIII, VEGFR1, VEGF or bFGFR.

In treating cancer according to the invention, one would contact the tumor cells with an agent in addition to the antisense construct. This may be achieved by irradiating the localized tumor site with radiation such as X-rays, UV-light, β-rays or even microwaves. Alternatively, the tumor cells may be contacted with the agent by administering to the subject a therapeutically effective amount of a pharmaceutical composition comprising a compound such as, adriamycin, 5-fluorouracil, etoposide, camptothecin, actinomycin-D, mitomycin C, or more preferably, tamoxifen. The agent may be prepared and used as a combined therapeutic composition, or kit, by combining it with an TIE-2, EDNRA, TGFβ3, TGFRβIII, VEGFR1, VEGF or bFGFR construct, as described above.

Agents that directly cross-link nucleic acids, specifically DNA, are envisaged to facilitate DNA damage leading to a synergistic, antineoplastic combination with TIE-2, EDNRA, TGFβ3, TGFRβIII, VEGFR1, VEGF or bFGFR. Agents such as cisplatin, and other DNA alkylating agents may be used. Cisplatin has been widely used to treat cancer, with efficacious doses used in clinical applications of 20 mg/m² for 5 days every three wk for a total of three courses. Cisplatin is not absorbed orally and must therefore be delivered via injection intravenously, subcutaneously, intratumorally or intraperitoneally.

Agents that damage DNA also include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered intravenously through bolus injections at doses ranging from 25-75 mg/m² at 21 day intervals for adriamycin, to 35-50 mg/m² for etoposide intravenously or double the intravenous dose orally.

Agents that disrupt the synthesis and fidelity of nucleic acid precursors and subunits also lead to DNA damage. A number of nucleic acid precursors have been developed for this purpose. Particularly useful are agents that have undergone extensive testing and are readily available, such as 5-fluorouracil (5-FU). Although quite toxic, 5-FU is applicable in a wide range of carriers, including topical. However intravenous administration with doses ranging from 3 to 15 mg/kg/day is commonly used.

Other factors that cause DNA damage and have been used extensively include γ-rays, X-rays, and/or the directed delivery of radioisotopes to tumor cells. Other forms of DNA damaging factors also are contemplated such as microwaves and UV-irradiation. It is most likely that all of these factors effect a broad range of damage to DNA, on the precursors of DNA, the replication and repair of DNA, and the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The skilled artisan is directed to “Remington's Pharmaceutical Sciences” 15th Edition, chapter 33, and in particular to pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA Office of Biologics standards.

The inventors propose that the regional delivery of TIE-2, EDNRA, TGFβ3, TGFRβIII, VEGFR1, VEGF or bFGFR constructs to patients with breast cancer will be a very efficient method for delivering a therapeutically effective gene to counteract the clinical disease. Similarly, chemo- or radiotherapy may be directed to a particular, affected region of the subject's body. Alternatively, systemic delivery of expression construct and/or the agent may be appropriate in certain circumstances, for example, where extensive metastasis has occurred.

In addition to combining TIE-2, EDNRA, TGFβ3, TGFRβIII, VEGFR1, VEGF or bFGFR-targeted therapies with chemo- and radiotherapies, it also is contemplated that combination with other gene therapies will be advantageous. For example, simultaneous targeting of therapies directed toward TIE-2, EDNRA, TGFβ3, TGFRβIII, VEGFR1, VEGF or bFGFR and p53, BRCA1 or BRCA2 mutations may produce an improved anti-cancer treatment. Any other tumor-related gene conceivably can be targeted in this manner, for example, p21, Rb, APC, DCC, NF-1, NF-2, p16, FHIT, WT-1, MEN-I, MEN-II, VHL, FCC, MCC, ras, myc, neu, raf, erb, src, fms, jun, trk, ret, gsp, hst, bcl and abl.

Formulations and Routes for Administration to Patients

Where clinical applications are contemplated, it will be necessary to prepare pharmaceutical compositions—antisense vectors, virus stocks, proteins, antibodies and drugs—in a form appropriate for the intended application. Generally, this will entail preparing compositions that are essentially free of pyrogens, as well as other impurities that could be harmful to humans or animals.

One generally will desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as innocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions normally would be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remnington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Portions of this work are recited in Hilsenbeck et al. (1999), the entire text of which is incorporated herein by reference.

Materials and Methods Utilized in Examples 1 Through 4

Tumors and Microarray Hybridization

MCF-7 tumors were inoculated into the mammary fat pads of athymic nude mice supplemented with an estrogen pellet as described previously (Osborne et al., 1985) until tumors arose. The estrogen pellets were removed and the animals were treated with tamoxifen. Tumor volumes then declined and remained stable for several months. Invariably, however, after initial growth suppression, the tumors became resistant and growth resumed. Animals were sacrificed at various times to obtain cells from estrogen-stimulated (ES) tumors prior to tamoxifen treatment, from tamoxifen-sensitive (TS) tumors during tamoxifen treatment but prior to acquired resistance, and from tamoxifen-resistant (TR) tumors after tumor growth had resumed.

RNA was prepared from these tumors (n=5 tumors per group) using RNeasy kits (Qiagen Inc., Valencia, Calif.), and mRNA was isolated on Dynabeads (Dyne, Oslo, Norway) according to manufacturer's instructions. The RNAs were pooled in each group and used to synthesize 32P-radiolabeled cDNAs for hybridization to the Atlas™ Human cDNA Expression Array 1 according to the manufacturer's instructions (CLONTECH Laboratories, Inc., 1997) with SuperScriptII RT (Gibco BRL, Gaithersburg, Md.). The CLONTECH Atlas™ Human cDNA Expression Array comprises a positively charged 8×12 cm nylon membrane, duplicately spotted with 200-600 BP cDNA fragments representing 588 genes and 21 housekeeping genes or control sequences (CLONTECH Laboratories, Inc., 1997). Genes are arrayed in six quadrants with genes of like function (i.e. oncogenes, assorted receptors, etc.) grouped together geographically. The hybridization data were collected with a Molecular Dynamics Phosphoimager™ (Sunnyvale, Calif.).

Western Blot Analysis

Pulverized, frozen tumors were manually homogenized in a 5% SDS solution. After boiling and microcentrifugation, clear supernatants were collected and the protein concentration was determined by the bicinchoninic acid method (Pierce, Rockford, Ill.) as previously described (Tandon et al., 1989). Twenty-five μg of protein were separated on an acrylamide denaturing gel and transferred by electroblotting onto nitrocellulose membranes (Schleicher & Schuell, Keene, N.H.). The blots were first stained with StainAll Dye (Alpha Diagnostic Intl., Inc., San Antonio, Tex.) to confirm uniform transfer of all samples, and then incubated in blocking solution [5% non-fat dry milk in Tris-buffered saline-Tween (TBST:50 mM Tris-HCL pH 7.5, 150 mM NaCl, 0.05% Tween-20)]. After brief washes with TBST, the filters then were reacted with primary antibodies to erk-2 (UBI, Lake Placid, N.Y.) or HSF-1 (Stressgen, Victoria, Canada) for 1 h at room temperature followed by extensive washes with TBST. Blots were then incubated with horseradish peroxidase-conjugated secondary antibody (Amersham, Arlington Heights, Ill.) for 1 h washed with TBST, and developed using the ECL procedure (Amersham).

Statistical Considerations

Each hybridization (m=3) resulted in expression values for 588 genes and 21 controls (putative housekeeping genes and negative controls). The controls, which were more difficult to quantitate reliably, were not included in the statistical analyses. Expression of the highest and lowest expressed genes on the array varied by 2-3 orders of magnitude. Logarithmic transformation of the raw data reduced this range and helped equalize variability. This also means that additive effects on the log scale can be interpreted as fold changes in actual expression.

Due to expense, limited amounts of RNA and other considerations, array studies usually have few replications and invariably have orders of magnitude more variables (genes and expressed sequence tags) than observations (hybridizations). Here, the roles of variables and observations were switched by treating each tumor type as a variable (m=3) and each expressed gene sequence as an observation (n=588).

Principal Components Analysis (PCA) of mean-centered log-transformed data, based on the variance-covariance matrix (Tatsuoka, 1971), was then used to standardize across the three hybridizations and to extract three new axes (components P1, P2, and P3), expressed as linear combinations of the original axes (ES, TS, and TR).

P1=A₁*ES+B₁*TS+C₁*TR

P2=A₂*ES+B₂*TS+C₂*TR

P3=A₃*ES+B₃*TS+C₃*TR

In PCA, the coefficients (A's, B's, C's) are chosen so that the first component (P1) explains the maximal amount of variance in the data. The second component (P2) is perpendicular to the first and explains the maximal residual squared variation, and the third component (P3) is perpendicular to the first two. Meaning was ascribed to the new axes by visual examination of the coefficients. In these array studies, P1 represents the average level of expression across the tumor types. P2 and P3 represent differences between tumor types. A bivariate analysis, which results in two new axes (P1 and P2), was also performed to compare TS and TR. The coefficients do not always have a biologically sensible interpretation, although the higher order components can still be used to identify outlier genes, regardless of interpretation.

P2 (and P3 in the higher-order analysis) were used to identify “outlier” genes that might represent true alterations in gene expression. In the bivariate PCA of TS vs. TR, a normal approximation was used to construct a 99% prediction region for P2 (i.e. 0±2.57*SDᵣ). A robust estimate of the standard deviation (SDᵣ=interquartile range/1.35) was used to reduce the variance inflating effects of outliers (Venables et al., 1994 ). Genes outside the region were identified for further study. Analogously, in a trivariate PCA (ES, TS, TR) a 99% bivariate normal prediction ellipse was computed (Tatsuoka, 1971; Anderson, 1958) for P2 vs. P3 and genes outside the ellipse were selected for investigation.

This “robust prediction interval” approach seems justified on the following basis. While the distribution of P1 is highly skewed, higher order components are roughly symmetric. When there is no differential expression, as in a bivariate analysis of two array hybridizations using the same pool of RNA, the higher order components are approximately normally distributed. In studies comparing different pools of RNA, where some genes may be differentially expressed, the observed distribution of each higher order component (P2, P3, etc.) should comprise a mixture of central (μ=0) and noncentral (μ≠0) distributions. A robust estimator that focuses on the middle of the observed distribution, which should represent primarily unaltered genes, was used to increase sensitivity to identify truly altered genes. The prediction level (99%), which is analogous to the specificity of a diagnostic test, was chosen arbitrarily as representing a reasonable balance between identifying too many spuriously “significant” genes, versus missing true alterations. For display purposes, the data was back-transformed by exponentiating P2 and P3 so that the data are shown as approximate fold-increases or decreases in expression.

The ability of this methodology to detect true alterations was examined in a small simulation study. Log transformed values from a hypothetical bivariate array study with 588 genes were generated to have a common log-normally distributed component for level of expression (i.e. exp(X)+8, where X˜N(μ=0, σ=6)), and independent normally distributed errors (i.e. logₑ(Control)=exp(X)+8+Y, logₑ(Experimental)=exp(X)+8+Z, where Y,Z˜N(μ=0, σ=0.17)). The distributional parameters were chosen to mimic data seen in real studies. A small percentage of truly altered genes (2% or 4%) were created by shifting the error distribution for the experimental member of the pair up or down (with 50% probability) to represent an average 2 or 2.5-fold change from baseline (i.e. logₑ(Experimental)=exp(X)+8+W, where W˜N(μ=±0.7, σ=0.17)). The generated data were then analyzed as described above, and the number of truly altered and spuriously-altered genes falling outside the 99% prediction region was tabulated. Each scenario was replicated 100 times and the results were summarized over all replications. All analyses were performed using SAS (Version 6.11, Cary, N.C.).

### Example 1

### Bivariate Analysis

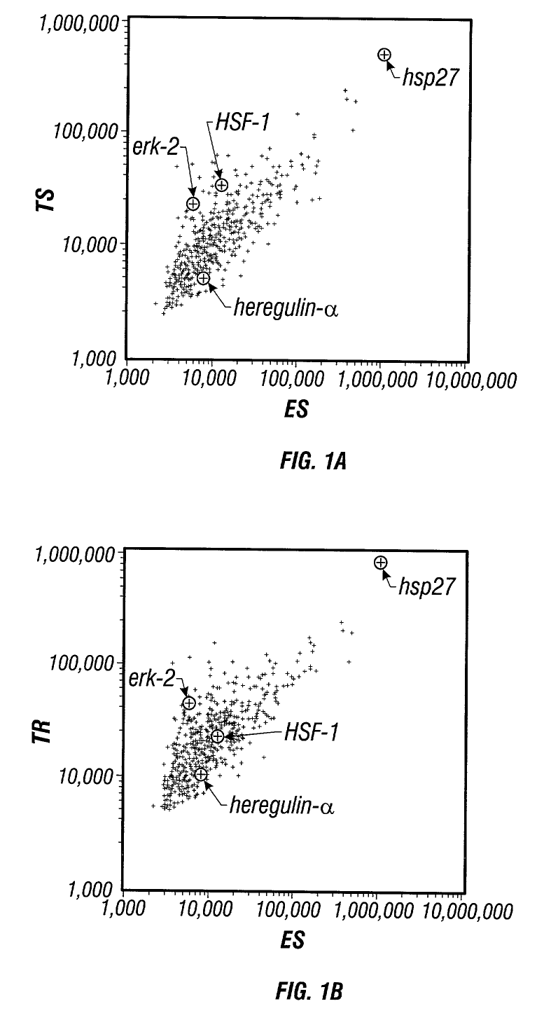

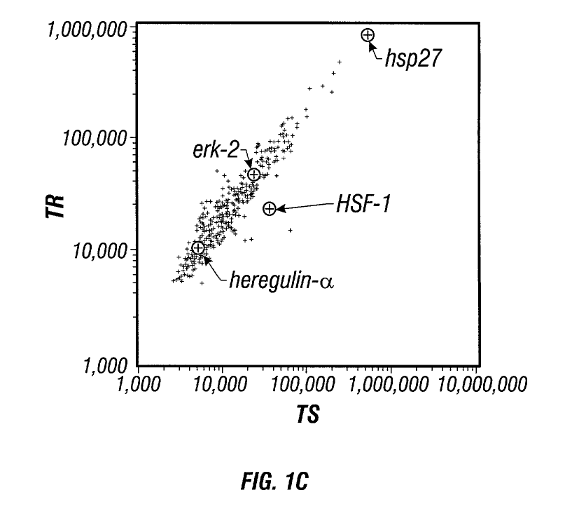

FIG. 1 shows the three bivariate log-log scatterplots that arise from pairwise comparisons of the data from the three hybridizations (ES, TS, TR). Each of the 588 genes on the array (excluding housekeeping and control genes) is represented by a point on the scatterplots. The individual values ranged over 2-3 orders of magnitude, indicating that the most highly expressed genes were expressed at 100 or 1,000-fold higher levels than the lowest expressed genes. For example, heat shock protein 27 (hsp27) was the most highly expressed gene on the array in all three tumor types. This is consistent with the previously published result that hsp27 is amplified and overexpressed in the late-passage MCF-7 cells used in this model (Fuqua et al., 1994). Similarly, the array results are consistent with previous findings (Tang et al., 1996) that heregulin α is expressed at relatively low levels in all three types of tumor cells.

In each scatterplot, most genes lie fairly close to a diagonal line of “identity”. This line may not be centered on the graph if there are differences in the average level of radioactivity of probes used in each hybridization. Distance along this line denotes differences in level of expression between genes, such as seen between hsp27 and heregulin a, while perpendicular distance away from the line denotes differences in expression within the same gene between tumor types.

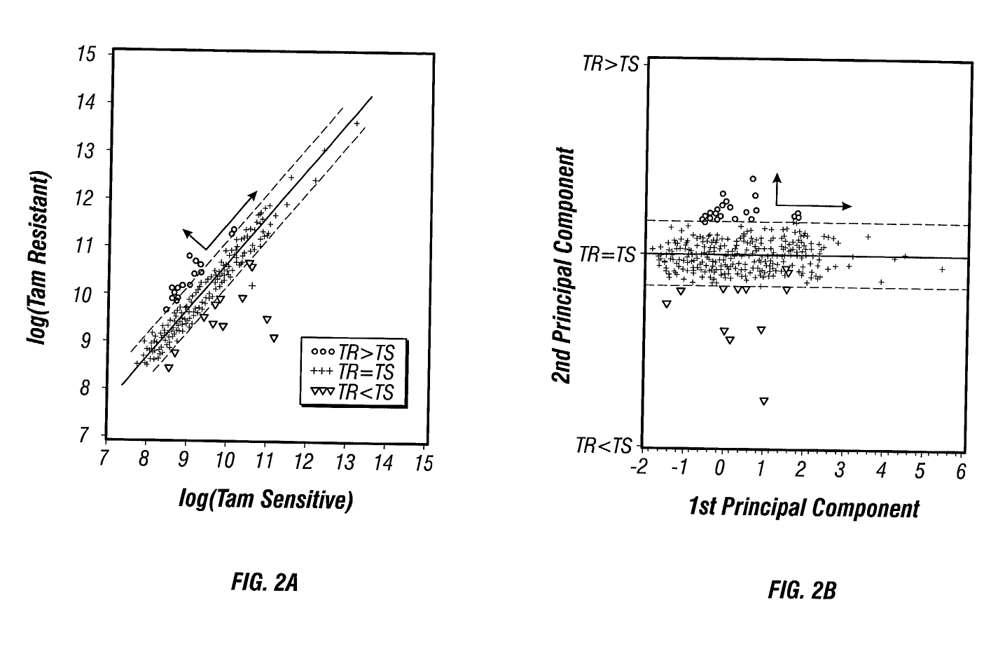

Principal Components Analysis (PCA) of the log-transformed expression data was used to produce a new set of axes (FIG. 2). For TS vs. TR tumors (FIG. 2A), the new x-axis or first principal component (P1) roughly corresponds to the line of “identity” and represents level of expression. The second principal component (P2) is perpendicular to the first, and represents difference in expression between tumor types. In the bivariate analysis, more than 97% of the total variation in the log-transformed data was associated with P1, leaving about 3% for P2. The two components are, by definition, not correlated (p=0). The distribution of P1 is skewed, as many genes on the array are expressed at low to moderate levels, while only a few are expressed at extremely high levels. The distribution of P2 is roughly symmetric, and a 99% robust prediction interval identified 35 outlier genes that may be over- or under-expressed in TR relative to TS tumors (FIG. 2B).

### Example 2

### Trivariate Analysis

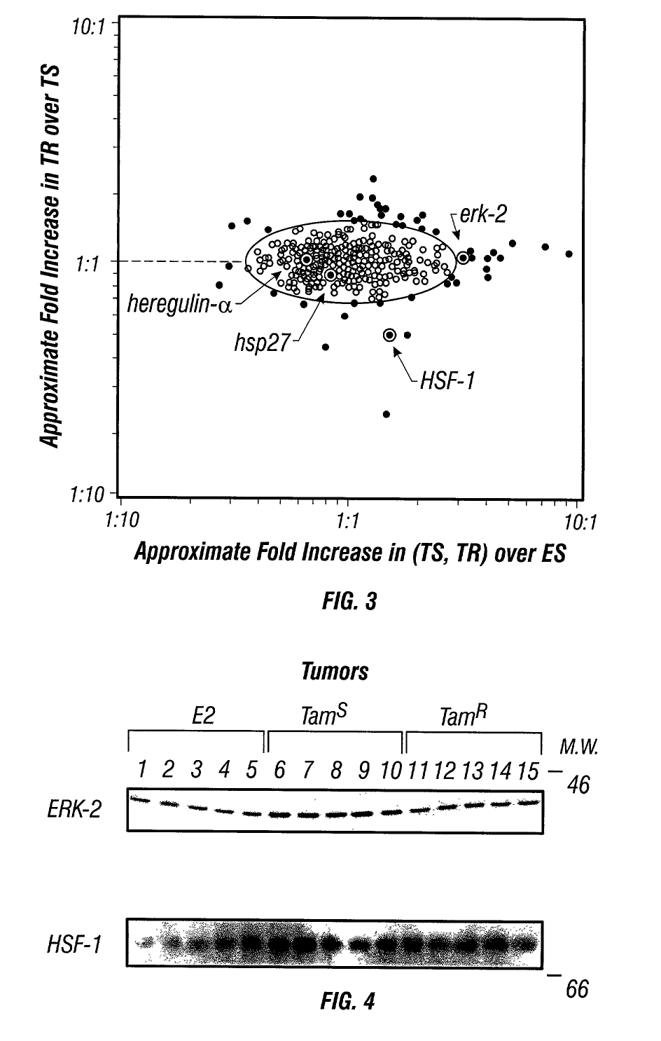

Bivariate PCA could be performed for each pair of tumor types, however, a more comprehensive three-way analysis is preferred and is more biologically relevant. PCA of the mean-centered log-transformed data (ES, TS, TR) yields three new axes (P1, P2, P3), which account for 90.5%, 8%, and 1.5% of the variation in the data, respectively. By inspection of the coefficients, the first principal component (P1) is again interpreted as the “average level of expression” since the coefficients were all positive and similar in value (0.63, 0.55, 0.55, respectively). The second principal component (P2) clearly contrasts ES to the average of TS and TR because the P2 coefficient for ES is negative (−0.78) and roughly equal to the sum of the TS and TR coefficients (0.46, 0.43, respectively). The third principal component (P3) represents primarily differences between TS and TR because the P3 coefficient for ES is small (0.02) and the TS and TR coefficients are nearly equal, but opposite in sign (0.69 and −0.72, respectively). FIG. 3 shows a scatterplot of P2 versus P3. Points near the center represent genes that were similarly expressed in all three tumor types while points on the periphery exhibit alterations in expression. Data have been back-transformed to show approximate fold changes in expression. A bivariate normal approximation with robust estimates of standard deviations was used to compute a 99% prediction ellipse. Genes lying outside the region may exhibit real alterations in level of expression that are associated with the biologic effects during the transition from ES to TS and TS to TR.

In addition, different regions of the P2×P3 plane correspond to different temporal patterns of expression alteration. For example, genes in the far right of FIG. 3 (i.e. near erk-2) are unregulated by tamoxifen relative to ES, but unchanged in TR relative to TS, while genes in the lower right (i.e. near HSF-1) are unregulated in TS relative to ES, but downregulated in TR tumors.

### Example 3

### Confirmation of Gene Expression by Western Blot Analysis

Two genes just outside of the 99% prediction ellipse (erk-2 and heat shock transcription factor 1 or HSF-1) were selected for quantitation by Western blot. These two were chosen based on their relatively low expression (FIG. 1) and modest alteration so that sensitivity questions could be addressed, and on the ready availability of specific antibodies. The erk-2 kinase is a known mediator of growth factor pathway signaling, and it has been shown that ER can activate its activity in MCF-7 cells (Migliaccio et al., 1996). HSF-1 is involved in cellular stress responses (Rabindran et al., 1991), and is thus a potential marker of tamoxifen-induced stress. The relative levels of erk-2 and HSF-1 predicted in the array study were indeed confirmed in an independent set of individual tumors (numbered 1-15 in FIG. 4) from the athymic nude mouse model. As predicted by FIG. 3A and FIG. 1A, Western blot results for HSF-1 indicate a significant upregulation in TS cells relative to ES, which is followed by down-regulation in TR to near ES levels (FIG. 4). Similarly for erk-2, there is a significant upregulation in TS relative to ES (FIG. 4) but relatively less change between TS and TR as reflected by the approximate fold increase in TR over TS around 1:1 (FIG. 4).

### Example 4

### Identification of Angiogenic Factors and Receptors as Markers for Tamoxifen-Resistant Breast Cancer

The techniques described in Examples 1-3 above were used to identify seven genes encoding angiogenic factors or angiogenic receptors as differentially expressed in tamoxifen-resistant breast cancer versus estrogen-stimulated or tamoxifen sensitive breast cancers, using the athymic mouse model and array screening to identify differentially expressed genes. Although angiogenic factors and receptors were known as a bad prognostic marker for breast cancer (Folkman, 1995a), this unexpected result is the first report of a correlation between expression levels for angiogenic factors and receptors and tamoxifen-resistant breast cancer.

The marker genes for tamoxifen-resistant breast cancer identified in the present application are tyrosine protein kinase receptor (TIE-2), endothelin-1 receptor (EDNRA), transforming growth factor β3 (TGFβ3), transforming growth factor receptor Om (TGFRβIII), vascular permeability factor receptor (VEGFR1), vascular endothelin growth factor (VEGF) and basic fibroblast growth factor receptor (bFGFR).

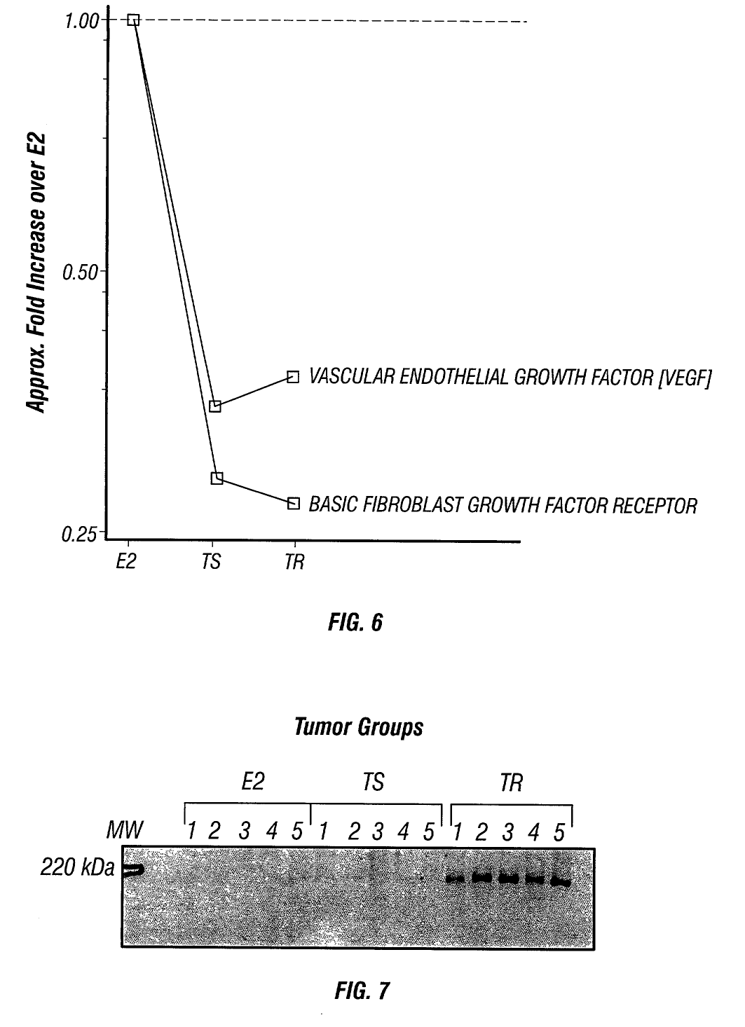

As shown in FIG. 6, both VEGF and bFGFR exhibited a decreased expression in breast cancers treated with tamoxifen. Expression was significantly inhibited in comparison with estrogen-stimulated breast cancer. While VEGF expression was significantly higher in tamoxifen-resistant compared to tamoxifen-sensitive breast cancers, no significant difference in bFGFR expression levels was observed between tamoxifen-sensitive and tamoxifen-resistant breast cancers.

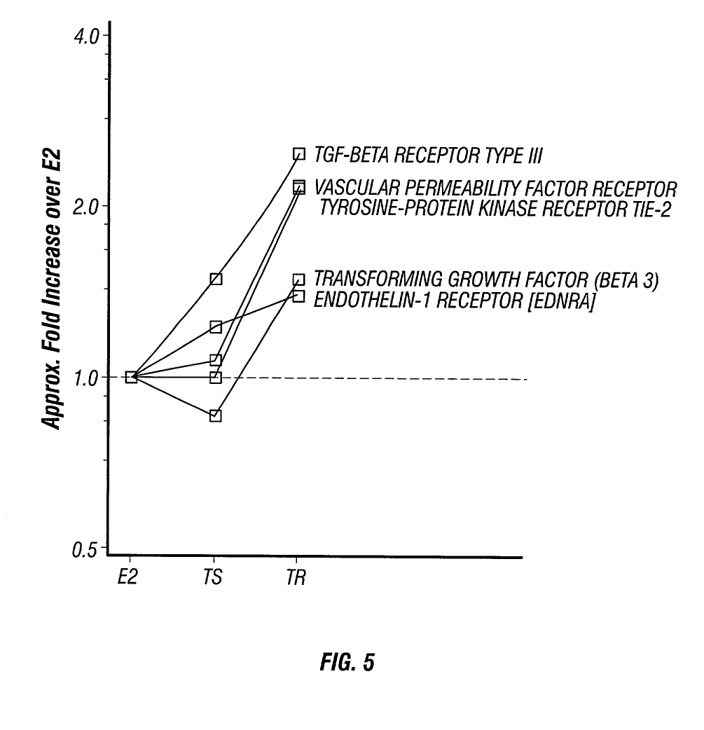

The remaining markers all showed a significant increase in expression in tamoxifen-resistant breast cancer, when compared to either estrogen-stimulated or tamoxifen-sensitive breast cancers. In FIG. 5, expression levels for TGFβ3, TIE-2, EDNRA, TGFβIII and VEGFR1 are elevated in tamoxifen-resistant (TR) tumors, compared to estrogen-stimulated (E2) or tamoxifen sensitive (TS) breast cancers.

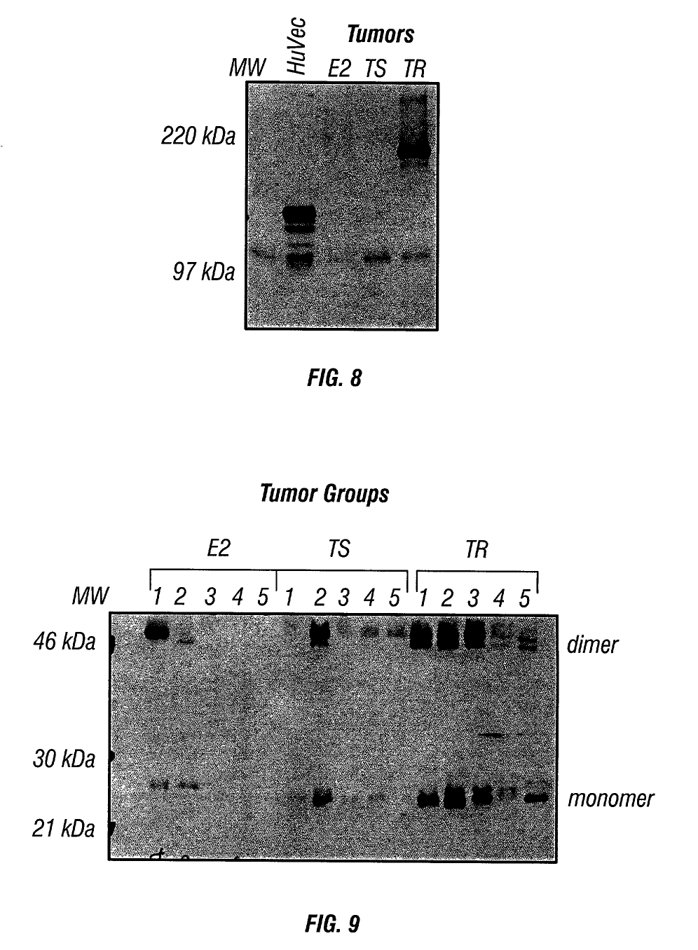

The results of array analysis were confirmed in part by Western blotting. As shown in FIGS. 7-9, both the TIE-2 and VEGF proteins showed increased expression in tamoxifen-resistant tumors, compared to tamoxifen-sensitive and estrogen-stimulated breast cancers. In addition, a higher molecular weight form of a putative TIE-2 related protein was observed only in TR tumors.

These results demonstrate that TIE-2, EDNRA, TGFβ3, TGFRβIII, VEGF and VEGFR 1 are all positive markers for tamoxifen-resistant breast cancer. Thus, assays for increased expression of these markers may be used to differentiate between tamoxifen-resistant and tamoxifen-sensitive forms of breast cancer, allowing more efficient clinical application of antiestrogen therapy. Significantly, these results suggest that antiangiogenic agents or treatment with antisense or “knock-out” constructs directed against these six genes may be used as adjuvants to tamoxifen treatment and can potentially be applied to convert tamoxifen-resistant breast cancers to tamoxifen-sensitive tumors. Further, application of antiangiogenic agents could potentially be used to prolong the sensitivity of tamoxifen-sensitive breast cancer to antiestrogen therapy. bFGFR may be important for angiogenesis to proceed but not necessarily a marker for tamoxifen resistance.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### References

The following literature citations as well as those cited above are incorporated in pertinent part by reference herein for the reasons cited in the above text.

Adamo et al., Endocrinology, 132:935-937, 1993.

Anderson, An Introduction to Multivariate Statistical Analysis, pg. 123, John Wiley & Sons, Inc., New York, 1958.

Augustin, Trends Pharmacol. Sci., 19: 216-222, 1998.

Baichwal and Sugden, In: Gene Transfer, Kucherlapati R, ed., New York, Plenum Press, pp. 117-148, 1986.

Barany and Merrifield, The Peptides, Gross and Meienhofer, eds., Academic Press, New York, pp. 1-284, 1979.

Bellus, J. Macromol. Sci. Pure Appl. Chem, A31(1): 1355-1376, 1994.

Benvenisty and Neshif, Proc. Nat. Acad. Sci. USA, 83:9551-9555, 1986.

Berns et al., Br. Cancer Res. Treat., 48: 87-92, 1998.

Campbell, In: Monoclonal Antibody Technology, Laboratory Techniques in Biochemistry and Molecular Biology, Burden and Von Knippenberg, Eds., Vol. 13:75-83, Elsevier, Amsterdam, 1984.

Capaldi et al., Biochem. Biophys. Res. Comm., 76:425, 1977

Chang et al., Hepatology, 14:124A, 1991.

Chee et al., Science 274: 610-614, 1996.

Chen and Okayama, Mol. Cell Biol., 7:2745-2752, 1987.

Chen et al., Genomics 51: 313-324, 1998.

CLONTECH Laboratories, Inc., Atlas cDNA Expression Arrays User Manual, CLONTECH Laboratories, Inc., Palo Alto, 1997.

Coffin, In: Virology, Fields et al., eds., Raven Press, New York, pp. 1437-1500, 1990.

Ccch et al., Cell, 27:487-496, 1981.

Couch et al., Am. Rev. Resp. Dis., 88:394-403, 1963.

Coupar et al., Gene, 68:1-10, 1988.

Coutts et al., J. of Cellular Biochem., 61: 1-11, 1996.

Cox et al., Breast Canc. Res. Treat. 32: 19-38, 1994.

Culver et al., Science, 256:1550-1552, 1992.

Davey et al., EP 329 822

DeRisi et al., Nature Genet. 14: 457-460, 1996.

Dubensky et al., Proc. Nat. Acad. Sci. USA, 81:7529-7533, 1984.

Dunn et al., J. of Steroid Biochem. and Mole. Biol., 46:469-479, 1993.

Elledge et al., Cancer Res. 54: 3752-3757, 1994

EPO No. 320 308

Fechheimer et al., Proc. Natl. Acad. Sci. USA, 84:8463-8467, 1987.

Ferkol et al., FASEB J., 7:1081-1091, 1993.

Fey and Penman, Proc. Natl. Acad. Sci. USA, 85:121-125, 1988.

Fodor et al., Science, 251:767-773, 1991.

Folkman, Breast Cancer Res. Treat., 36: 109-118, 1995a.

Folkman, In: The Molecular Basis of Cancer (Mendelsohn et al., eds.), pp. 206-232, WB Saunders, Philadelphia, 1995b.

Forster and Symons, Cell, 49:211-220, 1987.

Fraley et al., Proc. Natl. Acad. Sci. USA, 76:3348-3352, 1979.

Freifelder, Physical Biochemistry Applications to Biochemistry and Molecular Biology, 2nd ed., Wm. Freeman and Co., New York, N.Y., 1982.

Freshner, Animal Cell Culture: A Practical Approach, 2nd ed., Oxford/New York, IRL Press, Oxford University Press, 1992.

Friedmann, Science, 244:1275-1281, 1989.

Frohman, In: PCR™ Protocols: A Guide To Methods And Applications, Academic Press, N.Y., 1990.

Fujimoto and Katzenellenbogen, Mol. Endocrinol. 8: 296-304, 1994.

Fuqua et al., Breast Cancer Research and Treatment, 32:177-186, 1994.

Fuqua et al., Cancer Res., 49:4126-4129, 1989.

Garrido et al., Eur. J. Biol., 237:653-659, 1996.

GB 2 202 328

Gefter et al., Somatic Cell Genet., 3: 231-236, 1977.

Gerlach et al., Nature (London), 328:802-805, 1987.

Ghosh-Choudhury et al., EMBO J., 6:1733-1739, 1987.

Ghosh and Bachhawat, In: Liver Diseases, Targeted Diagnosis and Therapy Using Specific Receptors and Ligands. Wu et al., eds., Marcel Dekker, New York, pp. 87-104, 1991.

Gingeras et al., WO 88/10315.

Goding, In: Monoclonal Antibodies: Principles and Practice, 2d ed., Academic Press, Orlando, Fla., pp. 60-61, and 71-74, 1986.

Gomez-Foix et al., J. Biol. Chem., 267:25129-25134, 1992.

Gopal., Mol. Cell Biol., 5:1188-1190, 1985.

Graham and Prevec, In: Methods in Molecular Biology: Gene Transfer and Expression Protocol, E. J. Murray, ed., Humana Press, Clifton, N.J., 7:109-128, 1991.

Graham and van der Eb, Virology, 52:456-467, 1973.

Graham et al., J. Gen. Virol., 36:59-72, 1977.

Green and Olson, Proc. Natl. Acad. Sci. USA, 87:1213-1217, 1990.

Grunhaus and Horwitz, Seminar in Virology, 3:237-252, 1992.

Gyapay et al., Nat. Genet., 7:246-339, 1994.

Hacia et al., Nature Genetics, 14:441-447, 1996.

Harland and Weintraub, J. Cell Biol., 101:1094-1099, 1985.

Harlow and Lane, Antibodies: A Laboratory Manual, Cold Spring Harbor Laboratory, Cold Spring Harbor, N.Y., 1988.

Hermonat and Muzycska, Proc. Natl. Acad. Sci. USA, 81:6466-6470, 1984.

Hersdorffer et al., DNA Cell Biol., 9:713-723, 1990.

Herz and Gerard, Proc. Natl Acad. Sci. USA, 90:2812-2816, 1993.

Hickey et al., Nucl. Acids Res., 14:4127-4145, 1986.

Hilsenbeck et al., J. Natl. Cancer Inst., 91: 453-459, 1999.

Hopp and Fuqua, J. Mammary Gland Biol. Neoplasia, 3: 73-83, 1998.

Horwich, et al., J. Virol., 64:642-650, 1990.

Ikeuchi, Cytogenet Cell Genet 38:56-61, 1984.

Innis et al., PCR Protocols, Academic Press, Inc., San Diego Calif., 1990.

Johnson et al., “Peptide Turn Mimetics,” In: Biotechnology And Pharmacy, Pezzuto et al., eds., Chapman and Hall, New York, 1993.

Johnston, Anti-Cancer Drugs, 8: 911-930, 1997.

Jones and Shenk, Cell, 13:181-188, 1978.

Joyce, Nature, 338:217-244, 1989.

Kaneda et al., Science, 243:375-378, 1989.

Karlsson et al., EMBO J., 5:2377-2385, 1986.

Kato et al., J. Biol. Chem., 266:3361-3364, 1991.

Khan et al., Cancer Res. 58: 5009-5013, 1998.

Kim and Cech, Proc. Natl. Acad. Sci. USA, 84:8788-8792, 1987.

Kinzler and Vogelstein, Nature 386: 761-763, 1997.

Klein et al., Nature, 327:70-73, 1987.

Kohler and Milstein, Nature, 256:495-497, 1975.

Kohler and Milstein, Eur. J. Immunol., 6: 511-519, 1976.

Kozak, Microbiological Reviews, 47:1-45, 1983.

Kwoh et al., Proc. Nat. Acad. Sci. USA, 86: 1173, 1989.

Kyte and Doolittle, J. Mol. Biol., 157(1):105-132, 1982.

Lavoie et al., J. of Biol. Chem., 268:3420-3429, 1993.

Le Gal La Salle et al., Science, 259:988-990, 1993.

Lee and Feinberg, Cancer Res., 57: 3131-3134, 1997.

Lee et al., Cancer Res. 58: 1140-1143, 1998.

Lemieux et al., Proc. Am. Assn. Cancer Res., 37:68, 1996.

Lemiux and Fuqua, J. Steroid Biochem. Molec. Biol., 56: 87-91, 1996.

Levrero et al., Gene, 101: 195-202, 1991.

Li et al., Cell 88: 143-154, 1997.

Lin et al., Proc. Natl. Acad. Sci. USA, 95: 8829-8834, 1998.

Mahvi et al., Endocrine, 4:269-275, 1996.

Mann et al., Cell, 33:153-159, 1983.

Markowitz et al., J. Virol., 62:1120-1124, 1988.

Marschall and Hodgson, Nature Biotechnol. 16: 27-31, 1998.

Merrifield, Science, 232: 341-347, 1986

Michel and Westhof, J. Mol. Biol. 216: 585-610, 1990.

Migliaccio et al., EMBO J., 15: 1292-1300, 1996.

Miki et al., Science 266: 66-71, 1994.

Miller et al., WO 89/06700

Moorehead et al., Exp. Cell Res. 20:613-616, 1960.

Moretti-Rojas et al., Breast Cancer Res. Treat, 11: 155-163, 1988.

Mulligan, Science, 260:926-932, 1993.

Myers, EP 0273085

Neuman et al., Mol. Cell. Biol., 17: 5338-5347, 1997.

Nicolas and Rubinstein, In: Vectors: A survey of molecular cloning vectors and their uses, Rodriguez and Denhardt, eds., Stoneham: Butterworth, pp. 494-513, 1988.

Nicolau and Sene, Biochim. Biophys. Acta, 721:185-190, 1982.

Nicolau et al., Methods Enzymol., 149:157-176, 1987.

Oesterreich et al., Biochemical and Biophysical Research Communications, 222:155-163, 1996a.

Oesteffeich et al., Cancer Research, 53:4443-4448, 1993.

Oesterreich et al., Clinical Cancer Research, 2:1199-1206, 1996b.

Oesterreich et al., J. Cell. Biochem., 67: 275-286, 1997.

Ohara et al., Proc. Nat'l Acad. Sci. USA, 86:5673-5677, 1989.

Osborne and Fuqua, Breast Cancer Res. Treat. 32: 49-55, 1994.

Osborne et al., Cancer Res., 45: 584-590, 1985.

Paskind et al., Virology, 67:242-248, 1975.

PCT/US87/00880

PCT/US89/01025

Pease et al., Proc. Natl. Acad. Sci. USA, 91:5022-5026, 1994.

Perales et al., Proc. Natl. Acad. Sci. 91:4086-4090, 1994.

Pignon et al., Hum. Mutat., 3: 126-132, 1994.

Potter et al., Proc. Nat. Acad. Sci. USA, 81:7161-7165, 1984.

Rabindran et al., Proc. Natl. Acad. Sci. USA, 88: 6906-6910, 1991.

Racher et al., Biotechnology Techniques, 9:169-174, 1995.

Ragot et al., Nature, 361:647-650, 1993.

Reinhold-Hurek and Shub, Nature, 357:173-176, 1992.

Reimington's Pharmaceutical Sciences, 15th ed., pp. 1035-1038 and 1570-1580.

Renan, Radiother. Oncol., 19:197-218, 1990.

Renz and Fackelmayer, Nucl. Acids Res., 24:843-849, 1996.

Rich et al., Hum. Gene Ther., 4:461-476, 1993.

Richards et al., Intl. J. Oncol., 8:1265-1271, 1996.

Ridgeway, In: Vectors: A Survey of Molecular Cloning Vectors and Their Uses, Rodriguez et al., eds., Stoneham: Butterworth, pp. 467-492, 1988.

Rippe et al., Mol. Cell Biol., 10:689-695, 1990.

Rosenfeld et al., Cell, 68:143-155, 1992.

Rosenfeld et al., Science, 252:431-434, 1991.

Roux et al., Proc. Nat'l Acad. Sci. USA, 86:9079-9083, 1989.

Sager, Science 246, 1406-1412, 1989.

Saiki et al., Science, 239:487-491, 1988.

Sambrook et al., In: Molecular Cloning: A Laboratory Manual, 2d Ed., Cold Spring Harbor Laboratory Press, Cold Spring Harbor, N.Y., 1989.

Samuel et al., J. Cell. Biochem. 66: 9-15, 1997.

Sarver, et al., Science, 247:1222-1225, 1990.

Scanlon et al., Proc Natl Acad Sci USA, 88:10591-10595, 1991.

Schena et al., Science, 270: 467-470, 1995.

Schiff et al., Specialized Program of Research Excellence (SPORE) Annual Meeting, July 13-14, Washington, D.C., 1998.

Seymor et al., British Journal of Cancer, 61:886-890, 1990.

Shena et al., Proc. Natl. Acad. Sci. USA, 93: 10614-10619, 1996.

Shepherd et al., Proc. Natl. Acad. Sci. USA, 91:2629-2633, 1994.

Shoemaker et al., Nature Genetics 14:450-456, 1996.

Slamon et al., Science 235; 177-182, 1987.

Sliutz et al., British Journal of Cancer, 74:172-177, 1996.

Stanbridge, Trends Biochem. Sci. 6: 213-219, 1990.

Stewart and Young, Solid Phase Peptide Synthesis, 2d. ed., Pierce Chemical Co., 1984.

Stratford-Perricaudet and Perricaudet, In: Human Gene Transfer, O. Cohen-Haguenauer et al., eds., John Libbey Eurotext, France, pp. 51-61, 1991.

Stratford-Pericaudet et al., Hum. Gene. Ther. 1:241-256, 1990.

Stratton and Wooster, Curr. Opin. Genet. Devel. 6: 93-97, 1996.

Tam et al., J. Am. Chem. Soc., 105:6442, 1983.

Tandon et al., Journal of Clinical Oncology, 7:1120-1128, 1989.

Tandon et al., Proc. Am. Soc. Clin. Oncol., 9:84, 1990.

Tang et al., Cancer Res., 56: 3350-3358, 1996.

Tatsuoka, Multivariate Analysis: Techniques for Educational and Psychological Research, John Wiley & Sons, Inc., New York, 1971.

Temin, In: Gene Transfer, Kucherlapati R, ed., New York, Plenum Press, pp. 149-188, 1986.

Thompson et al., Br. J. Cancer, 63: 609-614, 1991.

Top et al., J. Infect. Dis., 124:155-160, 1971.

Trink et al., Oncogene 16: 2815-2818, 1998.

Tur-Kaspa et al., Mol. Cell Biol., 6:716-718, 1986.

U.S. Pat. No. 5,279,721

U.S. Pat. No. 4,196,265

U.S. Pat. No. 4,883,750

U.S. Pat. No. 4,683,195

U.S. Pat. No. 4,683,202

U.S. Pat. No. 4,800,159

U.S. Pat. No. 5,354,855

Varmus et al., Cell, 25:23-36, 1981.

Venables et al., Modern Applied Statistics with S-Plus, Springer-Verlag, New York, 1994.

Wagner et al., Science, 260:1510-1513, 1990.

Walker et al., Proc. Natl. Acad. Sci. USA, 89:392-396 1992.

Welford et al., Nucleic Acids Res. 26: 3059-3065, 1998.

WO 90/07641, filed Dec. 21, 1990.

Wong et al., Gene, 10:87-94, 1980.

Wright and Manos, In: PCR™ protocols, Innes et al. (eds.), Academic Press, San Diego, pp. 153-158, 1990.

Wu and Wu, Adv. Drug Delivery Rev., 12: 159-167, 1993.

Wu and Wu, Biochemistry, 27: 887-892, 1988.

Wu and Wu, J. Biol. Chem., 262: 4429-4432, 1987.

Wu et al., Genomics, 4:560, 1989.

Yanagisawa et al., Cancer Res., 56:457-462, 1996.

Yang et al., Proc. Natl. Acad. Sci. USA, 87:9568-9572, 1990.

Zhang et al., Cancer Res., 57: 1244-1249, 1997a.

Zhang et al., Science, 276: 1268-1272, 1997b.

1. A method for detecting tamoxifen-resistant breast cancer cells, comprising:
a) obtaining a sample suspected of containing tamoxifen-resistant breast cancer cells;
b) contacting said sample with an antibody that specifically binds to a polypeptide selected from the group consisting of tyrosine protein kinase receptor (TIE-2), endothelin-1 receptor (EDNRA), transforming growth factor β3 (TGFβ3), transforming growth factor receptor βIII (TGFRβIII), vascular permeability factor receptor (VEGFR1), vascular endothelin growth factor (VEGF) and basic fibroblast growth factor receptor (bFGFR), under conditions effective to bind said antibody and form a complex;
c) measuring the amount of said polypeptide present in said sample by quantitating the amount of said complex; and
d) comparing the amount of polypeptide present in said sample with the amount of polypeptide in estrogen-stimulated, tamoxifen-sensitive and tamoxifen-resistant breast cancer cells, wherein an increase in the amount of TIE-2, EDNRA, TGFβ3, TGFRβIII, VEGF or VEGFR1 polypeptide or a decrease in the amount of bFGFR polypeptide in said sample compared with the amount in estrogen-stimulated or tamoxifen-sensitive breast cancer cells indicates the presence of tamoxifen-resistant breast cancer cells.

2. The method of claim 1, further comprising:
a) measuring the amounts of two or more polypeptides selected from the group consisting of TIE-2, EDNRA, TGFβ3, TGFRβIII, VEGFR1, VEGF and bFGFR; and
b) for each polypeptide, comparing the amount of said polypeptide present in said sample with the amount of the same polypeptide present in estrogen-stimulated, tamoxifen-sensitive and tamoxifen-resistant breast cancer cells.

3. The method of claims 1 or 2, further comprising providing a diagnosis of tamoxifen-sensitive or tamoxifen-resistant breast cancer.

4. The method of claims 1 or 2, further comprising providing a prediction of the existence or development of tamoxifen-resistant breast cancer.

5. A method of determining survival for an individual with breast cancer, comprising determining the levels of TIE-2, EDNRA, TGFβ3, TGFRβIII, VEGFR1, VEGF or bFGFR polypeptide in a breast cancer tissue sample from said individual, wherein the presence of elevated levels of TIE-2, EDNRA, TGFβ3, TGFRβIII, VEGF or VEGFR1 polypeptide or decreased levels of bFGFR polypeptide in said tissue sample relative to estrogen-stimulated or tamoxifen sensitive breast cancer samples is associated with a decreased survival of the individual.

6. A method for detecting tamoxifen-resistant breast cancer cells, comprising:
a) isolating a nucleic acid from a sample suspected of containing tamoxifen-resistant breast cancer cells;
b) contacting said nucleic acid with a pair of primers effective to amplify the nucleic acid sequences of TIE-2, EDNRA, TGFβ3, TGFRβIII, VEGFR1, VEGF or bFGFR;
c) amplifying said nucleic acid using said primers to form an amplification product;
d) quantitating the amount of said amplification product formed; and
e) comparing the amount of said amplification product formed from said sample with the amount of amplification product formed under identical conditions from estrogen-stimulated, tamoxifen-sensitive and tamoxifen-resistant breast cancer cells, wherein a difference in the amount of said amplification product formed from said sample compared with the amount formed from estrogen-stimulated or tamoxifen-sensitive breast cancer cells indicates the presence of tamoxifen-resistant breast cancer cells.

7. The method of claim 6, further comprising:
a) measuring the amount of two or more amplification products using primers effective to amplify the nucleic acid sequences of TIE-2, EDNRA, TGFβ3, TGFRβIII, VEGFR1, VEGF or bFGFR; and
b) for each amplification product, comparing the amount of amplification product formed from said sample with the amount of amplification product formed from estrogen-stimulated, tamoxifen-sensitive and tamoxifen-resistant breast cancer cells.

8. The method of claim 6 or 7, further comprising providing a diagnosis of tamoxifen-sensitive or tamoxifen-resistant breast cancer.

9. The method of claim 6 or 7, further comprising providing a prediction for likelihood of development of tamoxifen-resistant breast cancer and subsequent patient survival.

10. A method of determining survival for an individual with breast cancer, comprising determining the levels of TIE-2, EDNRA, TGFβ3, TGFRβIII, VEGFR1, VEGF or bFGFR amplification product formed from a breast cancer tissue sample from said individual, wherein the presence of elevated levels of TIE-2, EDNRA, TGFβ3, TGFRβIII, VEGF or VEGFR1 amplification product or decreased levels of bFGFR amplification product formed from said tissue sample compared with estrogen-stimulated or tamoxifen-sensitive breast cancer cells is associated with a decreased survival of the individual.

11. A method for altering the phenotype of a breast cancer cell comprising contacting the cell with (i) a gene selected from the group consisting of TIE-2, EDNRA, TGFβ3, TGFRβIII, VEGFR1, VEGF and bFGFR and (ii) a promoter active in said cancer cell, wherein said promoter is operably linked to the region encoding said gene, under conditions effective for the uptake and expression of said gene by said breast cancer cell.

12. A method for treating breast cancer, comprising:
a) providing an effective amount of an antiangiogenic agent; and
b) providing an effective amount of tamoxifen.

13. The method of claim 12, wherein the antiangiogenic agent is selected from the group consisting of AGM-1470 (TNP-470), platelet factor 4 and angiostatin.

14. A method for treating breast cancer, comprising:
a) providing an effective amount of an antisense construct containing a gene selected from the group consisting of TIE-2, EDNRA, TGFβ3, TGFRβIII, VEGF and VEGFR1 under conditions allowing for the uptake and expression of said construct by said breast cancer; and
b) providing an effective amount of tamoxifen.

15. A method for treating breast cancer, comprising:
a) providing an effective amount of an expression construct containing a gene encoding bFGFR under conditions allowing for the uptake and expression of said construct by said breast cancer; and
b) providing an effective amount of tamoxifen.

16. A kit comprising:
a) one or more antibodies that specifically bind to TIE-2, EDNRA, TGFβ3, TGFRβIII, VEGFR1, VEGF or bFGFR polypeptide; and
b) a container for each of said antibodies.

17. A kit comprising:
a) one or more pairs of primers effective to amplify the nucleic acid sequences of messenger RNAs encoded by TIE-2, EDNRA, TGFβ3, TGFRβIII, VEGFR1, VEGF or bFGFR; and
b) a container for each of said primers.

18. A method of detecting markers for tamoxifen-resistant breast cancer, comprising:
a) isolating nucleic acids from samples of estrogen-stimulated, tamoxifen-sensitive and tamoxifen-resistant breast cancers;
b) converting messenger RNAs to cDNAs;
c) screening the cDNA species with a human cDNA expression array; and
d) identifying cDNA species that are differentially expressed in tamoxifen resistant breast cancers versus estrogen-stimulated or tamoxifen sensitive breast cancers, wherein differential expression indicates a marker for tamoxifen-resistant breast cancer.

19. A pharmaceutical composition comprising two or more nucleic acids selected from the group consisting of TIE-2, EDNRA, TGFβ3, TGFRβIII, VEGFR1, VEGF and bFGFR.

20. The composition of claim 19, wherein said nucleic acids are in the form of vectors.

21. A pharmaceutical composition comprising two or more polypeptides selected from the group consisting of TIE-2, EDNRA, TGFβ3, TGFRβIII, VEGFR1, VEGF and bFGFR.
